(19) United States
(12) Patent Application Publication
(10) Pub. No.: US 2021/0276958 A1
(43) Pub. Date: Sep. 9, 2021

(54) METHOD FOR PRODUCING SUBSTITUTED N-ARYL PYRAZOLES

(71) Applicant: Bayer Aktiengesellschaft, Leverkusen (DE)

(72) Inventors: Andreas REMBIAK, Bad Soden (DE); Thomas MECHLER, Eschborn (DE); Mark James FORD, Niedernhausen (DE)

(73) Assignee: Bayer Aktiengesellschaft, Leverkusen (DE)

(21) Appl. No.: 17/058,035

(22) PCT Filed: May 20, 2019
(86) PCT No.: PCT/EP2019/062922

§ 371 Date: Nov. 23, 2020

(30) Foreign Application Priority Data
May 24, 2018 (EP) 18174045.7

(51) Int. Cl. C07D 231/12; C07D 307/20; C07C 243/28

(52) U.S. Cl. CPC C07D 231/12; C07C 243/28; C07D 307/20

(57) ABSTRACT

The present invention relates to a process for preparing compounds of the formula (I)

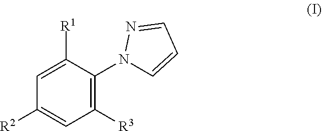

starting from compounds of the formula (II)

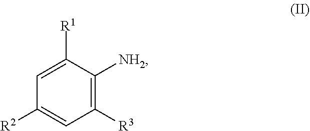

in which R¹, R² and R³ have the abovementioned meaning and where R¹ and R³ are not simultaneously hydrogen in any compound.

The invention further provides the compounds of the formulae (IVa), (IVb), (V) and (VI) in which R¹, R², R³, R⁵, M and n have the abovementioned meaning.

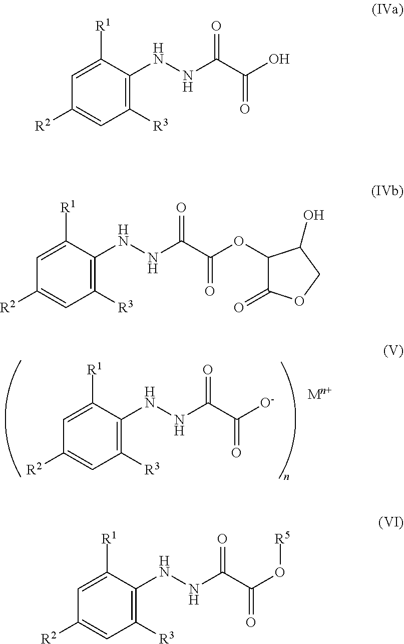

The present invention relates to a process for preparing compounds of the formula (I)

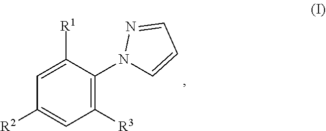

starting from compounds of the formula (II)

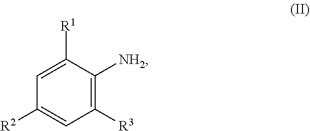

in which R¹, R² and R³ have the meanings described below.

One possible process for preparing compounds of formula (I) or precursors thereof is described for example in US2003/187233, WO2015/067646, WO2016/174052 and WO2015/067646. The preparation is performed by diazotization with sodium nitrite in aqueous hydrochloric acid or under anhydrous conditions in acetic acid and sulfuric acid, and subsequent reduction with tin(II) chloride and isolation of the hydrazine hydrochloride, which is cyclized in the following step under acidic conditions. Disadvantages in this method are the use of stoichiometric heavy-metal salts for the reduction step, and the isolation of a potentially toxic and to some extent unstable hydrazine salt.

The use of ascorbic acid as a possible reducing agent of diazonium salts has been described to date for the Fischer indole synthesis starting from electron-rich anilines (WO2005/103035, Org. Proc. Res. Dev. 2011, 15, 98) and in the synthesis of highly polar aminopyrazoles (US2002/0082274, RSC Adv. 2014, 4, 7019) under highly aqueous conditions. Furthermore, Chemistry—A European Journal, 23 (39), 2017, 9407 and Molecules, 21 (918), 2016, 1, describes the use of ascorbic acid for reducing aryldiazonium salts under highly aqueous conditions. Molecules, 21 (918), 2016, 1 additionally also describes problems in the reaction regime and also increased formation of secondary components at higher aniline concentration. The anilines used in the prior art, however, have a less complex substitution pattern on the aryl ring with lower lipophilicity compared to the compounds according to the invention. As a result, the compounds arising according to the invention have distinctly different polarities and thus also, for example, modified solubilities, including in aqueous hydrochloric acid or under highly aqueous conditions. These modified properties decisively influence the course of the reaction. Thus, a reaction regime under highly aqueous conditions as described in the prior art is disadvantageous for the process according to the invention, and the processes described there cannot easily be applied to the present objective.

N-Arylpyrazole derivatives are of great significance as a building block for synthesizing novel agrochemical active ingredients. The object of the present invention was therefore that of providing a process for preparing compounds of the general formula (I) which can be used industrially and cost-effectively and avoids the above-described disadvantages. It is also desirable to obtain the specific N-arylpyrazole derivatives with high yield and high purity, such that the target compound preferably does not have to be subjected to any further potentially complex purification.

This object was achieved according to the invention by a process for preparing compounds of the formula (I)

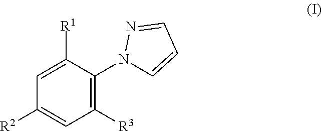

in which
R¹ is hydrogen, cyano, halogen, C₁-C₄-alkyl optionally substituted by halogen or CN, or C₁-C₄-alkoxy optionally substituted by halogen,
R² is trifluoromethylsulfonyl, trifluoromethylsulfinyl, trifluoromethylsulfanyl, halogen, C₁-C₄-alkyl optionally substituted by halogen, or C₁-C₄-alkoxy optionally substituted by halogen and
R³ is hydrogen, cyano, halogen, C₁-C₄-alkyl optionally substituted by halogen or CN, or C₁-C₄-alkoxy optionally substituted by halogen,
   where R¹ and R³ are not simultaneously hydrogen in any compound,

starting from compounds of the formula (II) in which R¹, R² and R³ have the abovementioned meaning and where R¹ and R³ are not simultaneously hydrogen in any compound,

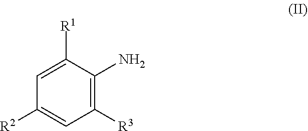

comprising the following steps (1) to (3)
(1) diazotization with compounds of the formula RNO₂ or M(NO₂)ₙ, where R is (C₁-C₆)-alkyl, n is one or two and M is ammonium, an alkali metal (with n=1) or an alkaline earth metal (with n=2), and at least one acid selected from mineral acids, sulfonic acids or carboxylic acids, wherein the carboxylic acids have a pKa of ≤2,
(2) reduction with ascorbic acid and
(3) cyclization with a 1,1,3,3-tetra(C₁-C₄)alkoxypropane in a polar solvent in the presence of at least one acid selected from mineral acids, sulfonic acids or carboxylic acids, where the carboxylic acids have a pKa ≤2.

The process according to the invention has the advantage over the previously described process that the use of stoichiometric heavy-metal salts and the waste resulting therefrom are dispensed with. In addition, the hydrazines are in the form of stable intermediates and are formed only as intermediates and in small quantities in the course of the reaction.

The preferred embodiments described below refer, if appropriate, to all formulae described herein.

In the context of the present invention, the term halogen preferably denotes chlorine, fluorine, bromine or iodine, particularly preferably chlorine, fluorine or bromine and very particularly preferably fluorine.

In one preferred embodiment of the invention,
R² is halogen-substituted C₁-C₄-alkyl or halogen-substituted C₁-C₄-alkoxy, such as for example difluoromethyl, trichloromethyl, chlorodifluoromethyl, dichlorofluoromethyl, trifluoromethyl, 1-fluoroethyl, 2-fluoroethyl, 2,2-difluoroethyl, 2,2,2-trifluoroethyl, 1,2,2,2-tetrafluoroethyl, 1-chloro-1,2,2,2-tetrafluoroethyl, 2,2,2-trichloroethyl, 2-chloro-2,2-difluoroethyl, 1,1-difluoroethyl, pentafluoroethyl, heptafluoro-n-propyl, heptafluoroisopropyl, nonafluoro-n-butyl, nonafluoro-sec-butyl, nonafluoro-tert-butyl, fluoromethoxy, difluoromethoxy, chlorodifluoromethoxy, dichlorofluoromethoxy, trifluoromethoxy, 2,2,2-trifluoroethoxy, 2-chloro-2,2-difluoroethoxy or pentafluoroethoxy.

Particularly preferably,
R² is fluorine-substituted C₁-C₄-alkyl or fluorine-substituted C₁-C₄-alkoxy.

Very particularly preferably,
R² is perfluoro-C₁-C₃-alkyl (CF₃, C₂F₅ or C₃F₇(n- or isopropyl)) or perfluoro-C₁-C₃-alkoxy (OCF₃, OC₂F₅ or OC₃F₇(n- or isopropoxy)).

Especially preferably,
R² is perfluoro-C₁-C₃-alkyl, such as trifluoromethyl, pentafluoroethyl, heptafluoroisopropyl or heptafluoro-n-propyl, especially heptafluoroisopropyl.

In one further preferred embodiment, R¹ and R³ in each case independently of one another are a substituent selected from hydrogen, Cl, Br, F, C₁-C₃-alkyl, halogen-substituted C₁-C₃-alkyl, C₁-C₃-alkoxy or halogen-substituted C₁-C₃-alkoxy.

According to the invention, R¹ and R³ are the substituents described herein, but R¹ and R³ are not simultaneously hydrogen in any compound. In other words, when R¹ in a compound is hydrogen, R³ is one of the other substituents described herein, and vice versa.

In one particularly preferred embodiment, R¹ and R³ in each case independently of one another are Cl, Br, C₁-C₃-alkyl, or fluorine-substituted C₁-C₃-alkyl, C₁-C₃-alkoxy or fluorine-substituted C₁-C₃-alkoxy, such as for example Cl, Br, methyl, trifluoromethyl, trifluoromethoxy or difluoromethoxy.

In one very particularly preferred embodiment, R¹ and R³ independently of one another are Cl, Br or F, especially Cl or Br.

In one particularly advantageous configuration of the invention, R¹ and R³ are the same halogen, especially chlorine.

In one preferred configuration of the invention, at least one of the radicals R¹, R², R³ is halogen-substituted C₁-C₄-alkyl or halogen-substituted C₁-C₄-alkoxy, particularly preferably fluorine-substituted C₁-C₃-alkyl or fluorine-substituted C₁-C₃-alkoxy.

In one further particularly advantageous configuration of the invention,

R¹ is halogen or C₁-C₃-alkyl, especially Br, Cl or methyl,

R² is fluorine-substituted C₁-C₄-alkyl or fluorine-substituted C₁-C₄-alkoxy, especially heptafluoroisopropyl, and

R³ is halogen, C₁-C₃-alkyl or fluorine-substituted C₁-C₃-alkyl, C₁-C₃-alkoxy or fluorine-substituted C₁-C₃-alkoxy, especially Cl, methyl, trifluoromethyl, trifluoromethoxy or difluoromethoxy.

The anilines of the formula (II) used as starting materials and the preparation thereof are known from the literature (e.g. EP2319830, US2002/198399, WO2006137395, WO2009030457, WO2010013567, WO2011009540).

Preference is given to the following anilines of the formula (II):
4-(1,1,1,2,3,3,3-heptafluoropropan-2-yl)-2,6-dimethylaniline
2,6-dichloro-4-(1,1,1,2,3,3,3-heptafluoropropan-2-yl)aniline
2-chloro-4-(1,1,1,2,3,3,3-heptafluoropropan-2-yl)-6-(trifluoromethyl)aniline
2-chloro-4-(1,1,1,2,3,3,3-heptafluoropropan-2-yl)-6-(trifluoromethoxy)aniline
2-chloro-6-(difluoromethoxy)-4-(1,1,1,2,3,3,3-heptafluoropropan-2-yl)aniline
4-(1,1,1,2,3,3,3-heptafluoropropan-2-yl)-2-methyl-6-(trifluoromethyl)aniline
2-bromo-4-(1,1,1,2,3,3,3-heptafluoropropan-2-yl)-6-(trifluoromethyl)aniline
2-bromo-4-(1,1,1,2,3,3,3-heptafluoropropan-2-yl)-6-(trifluoromethoxy)aniline

Particular preference is given here to the following compounds:
2,6-dichloro-4-(1,1,1,2,3,3,3-heptafluoropropan-2-yl)aniline
2-chloro-4-(1,1,1,2,3,3,3-heptafluoropropan-2-yl)-6-(trifluoromethyl)aniline
2-chloro-4-(1,1,1,2,3,3,3-heptafluoropropan-2-yl)-6-(trifluoromethoxy)aniline
2-chloro-6-(difluoromethoxy)-4-(1,1,1,2,3,3,3-heptafluoropropan-2-yl)aniline
2-bromo-4-(1,1,1,2,3,3,3-heptafluoropropan-2-yl)-6-(trifluoromethyl)aniline
2-bromo-4-(1,1,1,2,3,3,3-heptafluoropropan-2-yl)-6-(trifluoromethoxy)aniline

Very particular preference is given to
2,6-dichloro-4-(1,1,1,2,3,3,3-heptafluoropropan-2-yl)aniline,
2-chloro-4-(1,1,1,2,3,3,3-heptafluoropropan-2-yl)-6-(trifluoromethyl)aniline,
2-chloro-4-(1,1,1,2,3,3,3-heptafluoropropan-2-yl)-6-(trifluoromethoxy)aniline and
2-bromo-4-(1,1,1,2,3,3,3-heptafluoropropan-2-yl)-6-(trifluoromethoxy)aniline.

The following preferred compounds of the formula (I) are correspondingly formed from these compounds:
1-[4-(1,1,1,2,3,3,3-heptafluoropropan-2-yl)-2,6-dimethylphenyl]-1H-pyrazole
1-[2,6-dichloro-4-(1,1,1,2,3,3,3-heptafluoropropan-2-yl)phenyl]-1H-pyrazole
1-[2-chloro-4-(1,1,1,2,3,3,3-heptafluoropropan-2-yl)-6-(trifluoromethyl)phenyl]-1H-pyrazole
1-[2-chloro-4-(1,1,1,2,3,3,3-heptafluoropropan-2-yl)-6-(trifluoromethoxy)phenyl]-1H-pyrazole
1-[2-chloro-6-(difluoromethoxy)-4-(1,1,1,2,3,3,3-heptafluoropropan-2-yl)phenyl]-1H-pyrazole
1-[4-(1,1,1,2,3,3,3-heptafluoropropan-2-yl)-2-methyl-6-(trifluoromethyl)phenyl]-1H-pyrazole
1-[2-bromo-4-(1,1,1,2,3,3,3-heptafluoropropan-2-yl)-6-(trifluoromethyl)phenyl]-1H-pyrazole
1-[2-bromo-4-(1,1,1,2,3,3,3-heptafluoropropan-2-yl)-6-(trifluoromethoxy)phenyl]-1H-pyrazole Particular preference is given here to:
1-[2,6-dichloro-4-(1,1,1,2,3,3,3-heptafluoropropan-2-yl)phenyl]-1H-pyrazole
1-[2-chloro-4-(1,1,1,2,3,3,3-heptafluoropropan-2-yl)-6-(trifluoromethyl)phenyl]-1H-pyrazole
1-[2-chloro-4-(1,1,1,2,3,3,3-heptafluoropropan-2-yl)-6-(trifluoromethoxy)phenyl]-1H-pyrazole
1-[2-chloro-6-(difluoromethoxy)-4-(1,1,1,2,3,3,3-heptafluoropropan-2-yl)phenyl]-1H-pyrazole
1-[2-bromo-4-(1,1,1,2,3,3,3-heptafluoropropan-2-yl)-6-(trifluoromethyl)phenyl]-1H-pyrazole
1-[2-bromo-4-(1,1,1,2,3,3,3-heptafluoropropan-2-yl)-6-(trifluoromethoxy)phenyl]-1H-pyrazole.

Very particular preference is given to
1-[2,6-dichloro-4-(1,1,1,2,3,3,3-heptafluoropropan-2-yl)phenyl]-1H-pyrazole,
1-[2-chloro-4-(1,1,1,2,3,3,3-heptafluoropropan-2-yl)-6-(trifluoromethyl)phenyl]-1H-pyrazole,
1-[2-chloro-4-(1,1,1,2,3,3,3-heptafluoropropan-2-yl)-6-(trifluoromethoxy)phenyl]-1H-pyrazole and
1-[2-bromo-4-(1,1,1,2,3,3,3-heptafluoropropan-2-yl)-6-(trifluoromethoxy)phenyl]-1H-pyrazole.

In the context of the present invention, unless defined differently elsewhere, the term “alkyl”, according to the invention, either on its own or else in combination with further terms, for example haloalkyl, is understood to mean a radical of a saturated, aliphatic hydrocarbon group which has 1 to 12, preferably 1 to 6 and particularly preferably 1 to 4 carbon atoms and may be branched or unbranched. Examples of C-Cn-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl.

The term “alkoxy”, either on its own or else in combination with further terms, for example haloalkoxy, is understood in the present case to mean an O-alkyl radical, where the term “alkyl” is as defined above.

According to the invention, unless defined differently elsewhere, the term “aryl” is understood to mean an aromatic radical having 6 to 14 carbon atoms, preferably phenyl, naphthyl, anthryl or phenanthrenyl, particularly preferably phenyl.

Halogen-substituted radicals, for example haloalkyl, are mono- or polyhalogenated up to the maximum number of possible substituents. In the case of polyhalogenation, the halogen atoms may be identical or different. Unless stated otherwise, optionally substituted radicals may be mono- or poly substituted, where the substituents in the case of polysubstitutions may be the same or different.

The ranges specified above generally or in preferred ranges apply correspondingly to the overall process. These definitions can be combined with one another as desired, i.e. including combinations between the respective preferred ranges.

Preference according to the invention is given to using processes in which there is a combination of the meanings and ranges specified above as being preferred.

Particular preference according to the invention is given to using processes in which there is a combination of the meanings and ranges listed above as being particularly preferred.

Very particular preference according to the invention is given to using processes in which there is a combination of the meanings and ranges specified above as being very particularly preferred.

Especially used according to the invention are processes in which there is a combination of the meanings and ranges specified above with the term “especially”.

Specifically used according to the invention are processes in which there is a combination of the meanings and ranges specified above with the term “specifically”.

Process Description

Step (1), Diazotization:

According to the invention, the compounds of the formula (II) are reacted with compounds of the formula RNO₂ or M(NO₂)ₙ, where R is (C₁-C₆)-alkyl, preferably methyl, ethyl, n-propyl, isopropyl, n-butyl, isobutyl, tert-butyl, n-pentyl or isopentyl, n is one or two and M is ammonium, an alkali metal, preferably Li, Na or K (in each case n=1), or an alkaline earth metal, preferably Mg, Ca or Ba (in each case n=2), and at least one acid selected from mineral acids, sulfonic acids or carboxylic acids, where the carboxylic acids have a pKa of ≤2.

According to the invention, preference is given here to using between 0.9 and 2.0 equivalents, particularly preferably between 1.0 and 1.5 equivalents, very particularly preferably between 1.0 and 1.2 equivalents, based on the total molar amount of the compounds of the formula (II) used, of the compounds of the formula RNO₂ or M(NO₂)ₙ. Although the use of larger excesses is chemically possible, it is not expedient from an economic point of view.

Preference is given here to using the nitrites in pure form or, in the case of M(NO₂)ₙ, in pure form or as an aqueous solution at concentrations of 10-80% by weight, particularly preferably in pure form or as an aqueous solution at concentrations of 20-60% by weight and very particularly preferably in pure form or as an aqueous solution at concentrations of 35-50% by weight.

Suitable nitrites RNO₂ or M(NO₂)ₙ are for example alkali metal nitrites or alkaline earth metal nitrites or ammonium nitrite and also (C₁-C₆)-alkyl nitrites. Preference is given to LiNO₂, NaNO₂, KNO₂, Mg(NO₂)₂, Ca(NO₂)₂, Ba(NO₂)₂, n-butyl nitrite, tert-butyl nitrite, n-pentyl nitrite or isopentyl nitrite, particular preference is given to LiNO₂, NaNO₂, KNO₂, tert-butyl nitrite or isopentyl nitrite, very particular preference is given to NaNO₂.

The nitrites may be used alone or in a combination of two or more nitrites.

According to the invention, preference is given to using the acid in amounts, based on the total molar amount of the compounds of the general formula (II) used, of between 1.0 and 20.0 equivalents, particularly preferably between 3.0 and 10.0 equivalents, very particularly preferably between 2.0 and 7.0 equivalents.

Preference is given here to using the acid in pure form or as an aqueous solution at concentrations of 10-99% by weight, particularly preferably in pure form or as an aqueous solution at concentrations of 20-80% by weight and very particularly preferably in pure form or as an aqueous solution at concentrations of 25-60% by weight.

Suitable acids are preferably selected in accordance with the invention from mineral acids, sulfonic acids and carboxylic acids, where the carboxylic acids have a pKa of ≤2.

According to the invention, the term “mineral acids” encompasses all inorganic acids not containing carbon, such as for example HF, HCl, HBr, HI, H₂SO₄, HNO₃, and H₃PO₄.

Suitable mineral acids are particularly preferably selected from HI, HBr, HCl, H₂SO₄ and H₃PO₄, very particularly preferably from H₂SO₄ and H₃PO₄, and H₂SO₄ is especially preferred.

According to the invention, the term “sulfonic acids” encompasses the optionally substituted arylsulfonic and alkylsulfonic acids generally known to those skilled in the art, such as for example methanesulfonic acid, trifluoromethanesulfonic acid, benzenesulfonic acid and para-toluenesulfonic acid.

Suitable sulfonic acids are particularly preferably selected from methanesulfonic acid, trifluoromethanesulfonic acid and para-toluenesulfonic acid, very particularly preferably from methanesulfonic acid and trifluoromethanesulfonic acid, and methanesulfonic acid is especially preferred.

According to the invention, the term “carboxylic acids” encompasses all carbon-containing acids generally known to those skilled in the art and containing at least one carboxyl group (—COOH), such as for example optionally substituted alkylcarboxylic and arylcarboxylic acids and also optionally substituted alkyldicarboxylic and aryldicarboxylic acids having a pKa of ≤2, preferably of ≤1.

Suitable carboxylic acids are particularly preferably selected from trifluoroacetic acid, dichloroacetic acid and trichloroacetic acid, and trifluoroacetic acid is very particularly preferred.

In one particularly preferred configuration of the present invention, the suitable acids are selected from HCl, H₂SO₄, H₃PO₄, methanesulfonic acid, trifluoromethanesulfonic acid, para-toluenesulfonic acid, trifluoroacetic acid, dichloroacetic acid or trichloroacetic acid, very particularly preferably from H₂SO₄, H₃PO₄, methanesulfonic acid, trifluoromethanesulfonic acid or trifluoroacetic acid, especially preferably from H₂SO₄ or methanesulfonic acid.

The acids may be used alone or in a combination of two or more acids.

Step (1) is preferably carried out in a suitable solvent. Examples of suitable solvents are: carboxylic acids (for example acetic acid, n-propanoic acid, n-butanoic acid), esters (such as for example ethyl acetate, (n- and iso)propyl acetate, butyl acetate), ethers (for example tetrahydrofuran (THF), 2-methyl-THF, diglyme, 1,2-dimethoxyethane (DME), 1,4-dioxane), nitriles (for example acetonitrile, propionitrile), amide solvents (for example N,N-dimethylformamide (DMF), N,N-dimethylacetamide (DMAC), N-methylpyrrolidone (NMP)), alcohols (for example methanol, ethanol, (n- and iso)propanol) and also dipolar aprotic solvents (for example DMSO) or mixtures of these stated solvents.

Preferred solvents are acetonitrile, acetic acid, ethyl acetate, THF, DMAC, DME, diglyme or 1,4-dioxane. Very particular preference is given to acetic acid and acetonitrile or mixtures of acetonitrile and acetic acid.

The diazotization (step (1)) is preferably carried out at an ambient temperature in the range from −10° C. to 80° C., particularly preferably in the range from 0° C. to 60° C., very particularly preferably in the range from −5° C. to 40° C.

The diazotization is preferably carried out in the region of standard pressure (1013 hPa), e.g. in the range from 300 hPa to 5000 hPa or from 500 hPa to 2000 hPa, preferably such as in the range of 1013 hPa±200 hPa.

The reaction time for the diazotization is preferably in the range of the metering time for the nitrite. The reaction is instantaneous. Those skilled in the art can estimate the metering time without problems based on experience. However, at least half an hour is preferred, particularly preferably the metering time is in the range from 0.5 h to 3 h, very particularly preferably from 0.25 to 1.5 h.

A diazonium salt of the formula (III) is preferably formed after step (1),

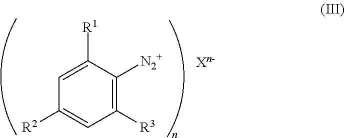

where R¹, R², R³ are as defined above, where R¹ and R³ are not simultaneously hydrogen in any compound, and Xⁿ⁻ according to the invention is a corresponding base, generally known to those skilled in the art, of the acids according to the invention from step (1), for example F₃CSO₃⁻, MeSO₃⁻, HSO₄⁻, SO₄²⁻ and H₂PO₄⁻, and n is 1 or 2.

Step (2), Reduction:

According to the invention, after step (1), a reduction with ascorbic acid is carried out in a further step (2).

In particular, this reduces the compounds of the formula (III) to give a reaction mixture comprising compounds of the formula (IVa) and/or (IVb)

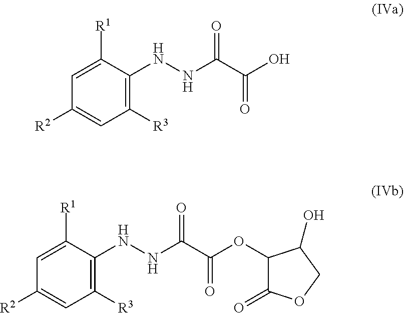

where R¹, R² and R³ are as defined above and where R¹ and R³ are not simultaneously hydrogen in any compound.

Preference is given here to using ascorbic acid in amounts of 0.9 to 2.0 equivalents, based on the total molar amount of the compound of the formula (II) used, particularly preferably between 1.0 and 1.5 equivalents, very particularly preferably between 1.0 and 1.2 equivalents.

Ascorbic acid can be used here as a solid or as an aqueous solution at concentrations of 5-40% by weight, preferably as a solid or an aqueous solution at concentrations of 10-30% by weight, very particularly preferably as a solid or an aqueous solution at concentrations of 15-25% by weight.

Ascorbic acid can be present in four stereoisomeric forms. The process according to the invention provides for the use both of one of the four pure isomeric ascorbic acids and of isomeric mixtures.

According to the invention, the addition of the ascorbic acid to the reaction mixture from step (1) can preferably take place in one portion or over a time period of 0.5-6 hours, particularly preferably in one portion or over a time period of 0.25-4 hours, very particularly preferably in one portion or over a time period of 0.5-3 hours. While a longer metering time is technically possible, this is not expedient from an economic point of view. According to the invention, the reduction preferably takes place without further dilution in the same solvent in which step (1) has already taken place.

According to the invention, the reduction can preferably take place by addition of ascorbic acid to a solution of the compounds of the general formula (III) in one of the solvents mentioned above under step (1) or by inverse metering.

The reduction reaction with ascorbic acid is preferably carried out at an ambient temperature in the range from −10° C. to 80° C., particularly preferably in the range from 0° C. to 60° C. and very particularly preferably in the range from −5° C. to 40° C.

The reaction is preferably carried out in the region of standard pressure (1013 hPa), e.g. in the range from 300 hPa to 5000 hPa or from 500 hPa to 2000 hPa, preferably such as in the range of 1013 hPa±200 hPa.

The reaction time for the reduction is preferably in the range from at least 5 min to 5 h, particularly preferably at least 15 min to 3 h and very particularly preferably at least 30 min to 2 h.

Step (2-a):

In one preferred configuration of the process according to the invention, after step (2), a base is added in a further step (2-a), as a result of which compounds of the formula (V) are precipitated out,

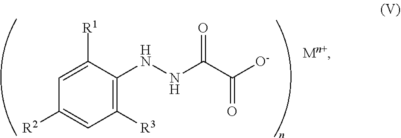

where R¹, R², R³ are as defined above, where R¹ and R³ are not simultaneously hydrogen in any compound, n is one or two and M is ammonium, an alkali metal, preferably Li, Na or K (in each case n=1), or an alkaline earth metal, preferably Mg, Ca or Ba (in each case n=2).

This process variant is particularly advantageous since these compounds have solubility behaviour in the commonly used solvents that is particularly favourable for the further processing, and these can therefore be obtained in particularly high purities and very good yields.

Suitable bases are for example carbonates (such as for example (NH₄)₂CO₃, Li₂CO₃, Na₂CO₃, K₂CO₃, CaCO₃, MgCO₃), hydrogencarbonates (such as for example NH₄HCO₃, LiHCO₃, NaHCO₃, KHCO₃), carboxylates (KOAc, NaOAc, LiOAc, KOOCH, NaOOCH, LiOOCH) or hydroxides (such as for example LiOH, NaOH, KOH). Preferably used according to the invention are hydrogencarbonates, especially NaHCO₃ or KHCO₃, carbonates, especially Na₂CO₃ or K₂CO₃, or hydroxides, especially NaOH or KOH, particularly preferably NaHCO₃, Na₂CO₃ or NaOH and very particularly preferably NaHCO₃ or NaOH, or mixtures of the bases mentioned.

The base is preferably used in amounts of between 1.0 and 5.0 equivalents (monoacidic bases) or between 0.5 and 2.5 equivalents (diacidic bases), based on the total molar amount of the compounds of the formula (II) used, particularly preferably between 1.2 and 3.0 equivalents (monoacidic bases) or between 0.6 and 1.5 equivalents (diacidic bases), very particularly preferably between 1.1 and 2.5 equivalents (monoacidic bases) or between 0.55 and 1.75 equivalents (diacidic bases).

For the less preferred case where step (2-a) takes place with step (1) and (2) in a “one-pot” reaction, the amounts of base have to be adjusted such that the acids present from these steps are firstly neutralized. This results in the following amounts of base:

The base in that case is preferably used in amounts of between 5 and 200 equivalents (monoacidic bases) or between 2.5 and 100 equivalents (diacidic bases), based on the total molar amount of the compounds of the formula (II) used, particularly preferably between 10 and 100 equivalents (monoacidic bases) or between 5 and 50 equivalents (diacidic bases), very particularly preferably between 20 and 60 equivalents (monoacidic bases) or between 10 and 30 equivalents (diacidic bases).

The base is preferably used in pure form or as an aqueous solution at concentrations of 1-70% by weight, particularly preferably as an aqueous solution at concentrations of 5-50% by weight, very particularly preferably as an aqueous solution at concentrations of 5-30% by weight.

Furthermore, the base is preferably added to a solution of the substance mixture from step 2, containing the products (IVa) and (IVb), in a suitable organic solvent. Preference is given to selecting a water-soluble organic solvent from the group of ethers (such as for example tetrahydrofuran (THF), 2-methyl-THF, diglyme, 1,2-dimethoxyethane (DME), 1,4-dioxane), nitriles (such as for example acetonitrile, propionitrile), amide solvents (such as for example DMF, DMAC, NMP), alcohols (such as for example methanol, ethanol, (n- and iso)propanol), ketones (such as for example acetone, ethyl methyl ketone) and also dipolar aprotic solvents (such as for example DMSO) or mixtures of these stated solvents. Particular preference is given to methanol, isopropanol, acetone, THF, DMAC and acetonitrile. Very particular preference is given to acetone.

The base is preferably added, according to the invention, while monitoring the pH, running through a pH range of between 1 and 10.

The reaction with base is preferably carried out at an ambient temperature in the range from 0° C. to 80° C., particularly preferably in the range from 15° C. to 60° C. and very particularly preferably in the range from 10° C. to 35° C.

The reaction is preferably carried out in the region of standard pressure (1013 hPa), e.g. in the range from 300 hPa to 5000 hPa or from 500 hPa to 2000 hPa, preferably such as in the range of 1013 hPa±200 hPa.

The reaction time for the salt formation to give compounds of the general formula (V) is preferably in the range from 0.5 h to 48 h, particularly preferably at least 3 h to 24 h and very particularly preferably 2 h to 12 h.

The compounds of the formula (V) are preferably isolated following the reaction by means of filtration and subsequent washing with water and also optionally finally using an organic, nonpolar aprotic solvent which is inert under the specific reaction conditions.

Examples of suitable organic, nonpolar aprotic solvents include: halohydrocarbons (e.g. chlorohydrocarbons, such as tetrachloroethane, dichloropropane, methylene chloride, 1,2-dichloroethane, dichlorobutane, chloroform, carbon tetrachloride, trichloroethane, trichloroethylene, pentachloroethane), halogenated aromatic hydrocarbons (e.g. difluorobenzene, chlorobenzene, bromobenzene, dichlorobenzene, chlorotoluene, trichlorobenzene), aliphatic, cycloaliphatic or aromatic hydrocarbons (e.g. pentane, hexane, heptane, octane, nonane and technical grade hydrocarbons, cyclohexane, methylcyclohexane, petroleum ether, ligroin, benzene, toluene, xylene, mesitylene, nitrobenzene), esters (e.g. methyl acetate, ethyl acetate, butyl acetate, isobutyl acetate, dimethyl carbonate, dibutyl carbonate, ethylene carbonate), ethers (e.g. diethyl ether, methyl tert-butyl ether, methyl cyclopentyl ether) or mixtures of the stated solvents. Particular preference is given to using dichloromethane, chlorobenzene, toluene, xylene, mesitylene, heptane, methylcyclohexane, ethyl acetate, methyl tert-butyl ether or methyl cyclopentyl ether, very particular preference is given to heptane, methyl tert-butyl ether, xylene or mesitylene.

The solvents may be used alone or in a combination of two or more.

Step (2-b):

In one preferred configuration of the process according to the invention, after step (2) or step (2-a), in a further step (2-b), at least one compound of the formula R⁵—OH is added, as a result of which, in the presence of at least one acid selected from mineral acids or sulfonic acids, compounds of the formula (VI) are formed,

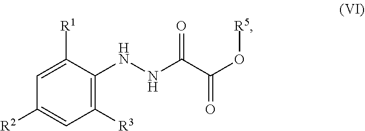

where R¹, R², R³ are as defined above, where R¹ and R³ are not simultaneously hydrogen in any compound, and R⁵ is C₁-C₄-alkyl.

Step (2-b) takes place in the presence of at least one acid selected from mineral acids or sulfonic acids. If a suitable acid is already present from step (1), and this was not removed during the process by purification or isolation of the intermediates, no further acid needs to be added. Otherwise, the acid is added afresh in step (2-b).

According to the invention, suitable acids are selected from mineral acids and sulfonic acids.

According to the invention, the term “mineral acids” encompasses all inorganic acids not containing carbon, such as for example HF, HCl, HBr, HI, H₂SO₄, HNO₃, and H₃PO₄.

Suitable mineral acids are particularly preferably selected from HI, HBr, HCl, H₂SO₄ and H₃PO₄, very particularly preferably from H₂SO₄, HBr and HCl, and H₂SO₄ is especially preferred.

According to the invention, the term “sulfonic acids” encompasses the optionally substituted arylsulfonic and alkylsulfonic acids generally known to those skilled in the art, such as for example methanesulfonic acid, trifluoromethanesulfonic acid, benzenesulfonic acid and para-toluenesulfonic acid.

Suitable sulfonic acids are particularly preferably selected from methanesulfonic acid, trifluoromethanesulfonic acid and para-toluenesulfonic acid, very particularly preferably from methanesulfonic acid and trifluoromethanesulfonic acid, and methanesulfonic acid is especially preferred.

In one particularly preferred configuration of the present invention, the suitable acids are selected from HCl, H₂SO₄, H₃PO₄, methanesulfonic acid, trifluoromethanesulfonic acid or para-toluenesulfonic acid, very particularly preferably from H₂SO₄, HCl, methanesulfonic acid or trifluoromethanesulfonic acid, especially preferably from H₂SO₄ or methanesulfonic acid.

The acids may be used alone or in a combination of two or more acids.

It is preferable according to the invention for the acid to be used as a pure substance or as a solution in a suitable organic solvent which is inert under the reaction conditions, especially in the solvent previously preferred for the reaction, preferably at a concentration of >30% by weight, particularly preferably at a concentration of >60% by weight. Particular preference is given, however, to using the acid as a pure substance and in the case of mineral acids in their commercially available concentrated form without further dilution.

Preference is given to adding the acid in step (2-b) in amounts, based on the total molar amount of the compounds of the general formula (II) used, of between 1.0 and 6.0 equivalents; particularly preferably 1.5 to 4.0 equivalents, very particularly preferably 1.2 to 3.0 equivalents, are used.

R⁵ in the compounds of the formula (VI) is (C₁-C₄)-alkyl, such as for example methyl, ethyl, n-propyl, isopropyl, n-butyl, 2-butyl or tert-butyl, preferably methyl or ethyl.

The alcohol R⁵—OH is preferably used simultaneously as solvent and reagent. The use of stoichiometric amounts of the alcohol R⁵—OH, based on the total molar amount used of compounds of the formula (II) in combination with solvents which are inert under the reaction conditions, such as for example toluene, xylene or chlorobenzene, is likewise possible according to the invention, but is less preferred.

Step (2-b) is preferably carried out at an ambient temperature in the range from 0° C. to 150° C., particularly preferably in the range from 10° C. to 100° C. and very particularly preferably in the range from 30° C. to 90° C.

The reaction is preferably carried out in the region of standard pressure (1013 hPa), e.g. in the range from 300 hPa to 5000 hPa or from 500 hPa to 2000 hPa, preferably such as in the range of 1013 hPa±200 hPa.

The reaction time for step (2-b) is preferably in the range from 0.5 h to 12 h, particularly preferably from 3 h to 8 h and very particularly preferably from 2 h to 7 h.

Reaction step (2-b) can follow step (2) or step (2-a).

Step (3), Cyclization:

The process according to the invention comprises, in a further step (3), the cyclization of the compounds obtained from step (2), (2-a) or (2-b) with 1,1,3,3-tetra(C₁-C₄)alkoxypropanes in a polar solvent and in the presence of at least one acid selected from mineral acids, sulfonic acids or carboxylic acids, where the carboxylic acids have a pKa ≤2.

Preference is given to using 1,1,3,3-tetramethoxypropane or 1,1,3,3-tetraethoxypropane; 1,1,3,3-tetramethoxypropane is particularly preferred. The 1,1,3,3-tetra(C₁-C₄)alkoxypropanes may be used alone or in a combination of two or more 1,1,3,3-tetra(C₁-C₄)alkoxypropanes.

The 1,1,3,3-tetra(C₁-C₄)alkoxypropanes are preferably added in amounts, based on the total molar amount of the compounds of formula (II) used, of 0.7 to 2.0 equivalents, particularly preferably of 0.9 to 1.5 equivalents and very particularly preferably of 0.8 to 1.1 equivalents. The use of larger excesses is not expedient from an economic point of view.

The 1,1,3,3-tetra(C₁-C₄)alkoxypropane compounds may be added in one portion or metered in. Preference is given to adding the 1,1,3,3-tetra(C₁-C₄)alkoxypropanes in one portion.

Suitable polar solvents for step (3) are the polar solvents commonly known to those skilled in the art, such as for example water, aqueous mineral acids, especially hydrochloric acid or sulfuric acid, carboxylic acids, in particular acetic acid, n-propanoic acid or n-butanoic acid, ethers, especially tetrahydrofuran (THF), 2-methyl-THF, diglyme, 1,2-dimethoxyethane (DME) or 1,4-dioxane, nitriles, especially acetonitrile or propionitrile, amide solvents, especially N,N-dimethylformamide (DMF), N,N-dimethylacetamide (DMAC) or N-methylpyrrolidone (NMP), alcohols, especially methanol, ethanol or (n- and iso)propanol, and also dipolar aprotic solvents (for example DMSO).

Preference is given to using aqueous hydrochloric acid, aqueous sulfuric acid, acetic acid, methanol or ethanol, particular preference is given to using methanol.

The solvents may be used individually or in a mixture of two or more.

In one preferred configuration of the process according to the invention, the compound R⁵—OH from step (2-b) serves as solvent for step (2-b) and step (3).

Step (3) takes place in the presence of at least one acid selected from mineral acids, sulfonic acids or carboxylic acids, where the carboxylic acids have a pKa ≤2.

If a suitable acid is already present from step (1) or (2-b), and this was not removed during the process by purification or isolation of the intermediates, no further acid needs to be added. Furthermore, no further acid needs to be added if step (3) is carried out in an aqueous mineral acid according to the invention or a carboxylic acid with pKa ≤2 as solvent.

Otherwise, the acid is added afresh in step (3). Suitable acids are selected according to the invention from mineral acids, sulfonic acids and carboxylic acids, where the carboxylic acids have a pKa of ≤2.

According to the invention, the term “mineral acids” encompasses all inorganic acids not containing carbon, such as for example HF, HCl, HBr, HI, H₂SO₄, HNO₃, and H₃PO₄.

Suitable mineral acids are particularly preferably selected from HI, HBr, HCl, H₂SO₄ and H₃PO₄, very particularly preferably from H₂SO₄, HBr and HCl, and H₂SO₄ is especially preferred.

According to the invention, the term “sulfonic acids” encompasses the optionally substituted arylsulfonic and alkylsulfonic acids generally known to those skilled in the art, such as for example methanesulfonic acid, trifluoromethanesulfonic acid, benzenesulfonic acid and para-toluenesulfonic acid.

Suitable sulfonic acids are particularly preferably selected from methanesulfonic acid, trifluoromethanesulfonic acid and para-toluenesulfonic acid, very particularly preferably from methanesulfonic acid and trifluoromethanesulfonic acid, and methanesulfonic acid is especially preferred. According to the invention, the term “carboxylic acids” encompasses all carbon-containing acids generally known to those skilled in the art and containing at least one carboxyl group (—COOH), such as for example optionally substituted alkylcarboxylic and arylcarboxylic acids and also optionally substituted alkyldicarboxylic and aryldicarboxylic acids, having a pKa of ≤2, preferably of ≤1.

Suitable carboxylic acids are particularly preferably selected from dichloroacetic acid, trichloroacetic acid and trifluoroacetic acid, and trifluoroacetic acid is very particularly preferred.

In one particularly preferred configuration of the present invention, the suitable acids are selected from HCl, H₂SO₄, H₃PO₄, methanesulfonic acid, trifluoromethanesulfonic acid, para-toluenesulfonic acid, trichloroacetic acid, dichloroacetic acid and trifluoroacetic acid, very particularly preferably from H₂SO₄, HCl, methanesulfonic acid, trifluoromethanesulfonic acid or trifluoroacetic acid, especially preferably from H₂SO₄ or methanesulfonic acid.

The acids may be used alone or in a combination of two or more acids.

It is preferable according to the invention for the acid to be used as a pure substance or as a solution in a suitable organic solvent which is inert under the reaction conditions, especially in the solvent previously preferred for the reaction, preferably at a concentration of >30% by weight, particularly preferably at a concentration of >60% by weight. Particular preference is given, however, to using the acid as a pure substance and in the case of mineral acids in their commercially available concentrated form without further dilution.

Preference is given to adding the acid in step (3) in amounts, based on the total molar amount of the compounds of the general formula (II) used, of between 1.0 and 6.0 equivalents; particularly preferably 1.5 to 4.0 equivalents, very particularly preferably 1.2 to 3.0 equivalents, are used.

The ring closure reaction with 1,1,3,3-tetra(C₁-C₄)alkoxypropane compounds is preferably carried out at an ambient temperature in the range from 0° C. to 100° C., more preferably in the range from 20° C. to 90° C., even more preferably in the range from 40° C. to 80° C.

The reaction is preferably carried out in the region of standard pressure (1013 hPa), e.g. in the range from 300 hPa to 5000 hPa or from 500 hPa to 2000 hPa, preferably such as in the range of 1013 hPa±200 hPa.

The reaction time for the ring closure reaction is preferably in the range from 0.05 h to 30 h, particularly preferably in the range from 0.5 h to 20 h, very particularly preferably in the range from 2 h to 15 h, especially in the range from 4 h to 8 h.

The workup and isolation of the compounds (I) may, after complete reaction, take place for example by removing the solvent, washing with water and extracting with a suitable organic solvent and separating the organic phase, and also removing the solvent under reduced pressure. The residue may furthermore be subjected to vacuum distillation at 0.05-1 mbar using a split-tube column and also crystallization in a solvent generally known to those skilled in the art.

The process according to the invention may comprise or consist of the following combinations of steps (1), (2), (2-a), (2-b) and (3):

step (1), step (2) and step (3),

step (1), step (2), step (2-a) and step (3),

step (1), step (2), step (2-b) and step (3),

step (1), step (2), step (2-a), step (2-b) and step (3).

In one preferred configuration of the process according to the invention, the process comprises the steps (1), (2), (2-a) and (3) or consists of these steps.

In a further preferred configuration of the process according to the invention, the process comprises the steps (1), (2), (2-b) and (3) or consists of these steps.

Particularly preferably, the process according to the invention comprises the steps (1), (2), (2-a), (2-b) and (3) or consists of these steps.

In one preferred configuration of the invention, steps (1) and (2) are carried out together in a “one-pot” reaction.

It is preferred in this configuration of the process according to the invention as a “one-pot” reaction that the diazonium salt (III) formed after step (1) from compound (II) is not isolated or purified.

It is furthermore preferred in this configuration of the process according to the invention as a “one-pot” reaction that neither the diazonium salt (III) formed after step (1) from compound (II) is isolated or purified, nor is there any essential removal and/or exchange of solvent.

It is furthermore preferred in this configuration of the process according to the invention as a “one-pot” reaction that neither the diazonium salt (III) formed after step (1) from compound (II) is isolated or purified, nor is there any essential removal and/or exchange of solvent, and steps (1) and (2) take place in the same reaction vessel. In this case, those skilled in the art will choose a reaction vessel from the start that can accommodate all volumes for reactions (1) and (2).

According to the invention, either step (2-a) can take place after isolation and optional purification of the substance mixture from step (2), or steps (1), (2) and (2-a) take place together in a “one-pot” reaction.

Preferably, step (2-a) takes place after isolation and optionally purification of the substance mixture from step (2).

In the case of the less-preferred configuration of the process according to the invention as a “one-pot” reaction, the diazonium salt (III) formed after step (1) from compound (II) and the product mixture formed after step (2) are not isolated or purified.

It is preferred in this configuration of the process according to the invention as a “one-pot” reaction that neither the diazonium salt (III) formed after step (1) from compound (II) and the product mixture formed after step (2) are isolated or purified, nor is there any essential removal and/or exchange of solvent.

It is furthermore preferred in this configuration of the process according to the invention as a “one-pot” reaction that neither the diazonium salt (III) formed after step (1) from compound (II) and the product mixture formed after step (2) are isolated or purified, nor is there any essential removal and/or exchange of solvent, and steps (1), (2) and (2-a) take place in the same reaction vessel. In this case, those skilled in the art will choose a reaction vessel from the start that can accommodate all volumes for reactions (1), (2) and (2-a).

In one further-preferred configuration of the invention, steps (2-b) and (3) are carried out together in a “one-pot” reaction.

Preference in this configuration of the process according to the invention is given to not isolating or purifying the compound (VI) formed after step (2-b).

In one specific configuration, the process according to the invention is characterized in that, after step (2) or step (2-a), in a further step (2-b), at least one compound of the formula R⁵—OH is added, as a result of which, in the presence of at least one acid selected from mineral acids or sulfonic acids, compounds of the formula (VI) are formed,

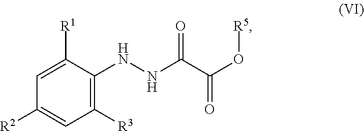

where R¹, R², R³ are defined according to Claim 1, where R¹ and R³ are not simultaneously hydrogen in any compound and R⁵ is C₁-C₄-alkyl and

in addition, the steps (2-b) and (3) are carried out together in a “one-pot” reaction, wherein the compound (VI) formed after step (2-b) is not isolated or purified.

It is furthermore preferred in the abovementioned configurations of the process according to the invention that neither the compound (VI) formed after step (2-b) is isolated or purified, nor is there any essential removal and/or exchange of solvent.

It is furthermore preferred in the abovementioned configurations of the process according to the invention that neither the compound (VI) formed after step (2-b) is isolated or purified, nor is there any essential removal and/or exchange of solvent, and steps (2-b) and (3) take place in the same reaction vessel. In this case, those skilled in the art will choose a reaction vessel from the start that can accommodate all volumes for reactions (2-b) and (3).

In a particularly preferred configuration of the invention, steps (1) and (2) are carried out as a “one-pot” reaction and steps (2-b) and (3) are also carried out as a “one-pot” reaction. The configurations respectively specified above as preferred for the individual “one-pot” reactions apply analogously.

In the process according to the invention, isolation and optionally purification of the product mixture from step (2) and/or of the compounds of the formula (V) after step (2-a) preferably takes place prior to the further conversion thereof.

During the reaction sequence in a “one-pot” reaction, it is possible to add reaction volumes in the form of solids, liquids or suspensions, for example in the form of solid, dissolved or suspended reducing agents, or solvent (the same solvent as in the first step or another solvent), but with the aim of a reaction sequence without essential/without exchange of solvent or active removal of solvent.

In other words, it is preferable for the reaction sequence to be a telescoped reaction in one or more vessels, preferably one vessel.

In the context of the present invention, the term “purification” refers to the enrichment of a substance (and therefore depletion of other substances) to a purity of at least 20% by weight (percent by weight of a substance based on the total mass measured. The proportion may be determined chromatographically for example (e.g. HPLC or gas chromatographically or gravimetrically)), preferably at least 50% by weight, even more preferably at least 75% by weight, e.g. 90% by weight, 98% by weight or greater than 99% by weight.

In one further preferred configuration of the process according to the invention, the compound R⁵—OH from step (2-b) serves as solvent for step (2-b) and step (3). Particular preference is given here to using the product mixture obtained from step 2 or the compounds of the formula (V) in a form dissolved in R⁵—OH, where R⁵ is as defined above.

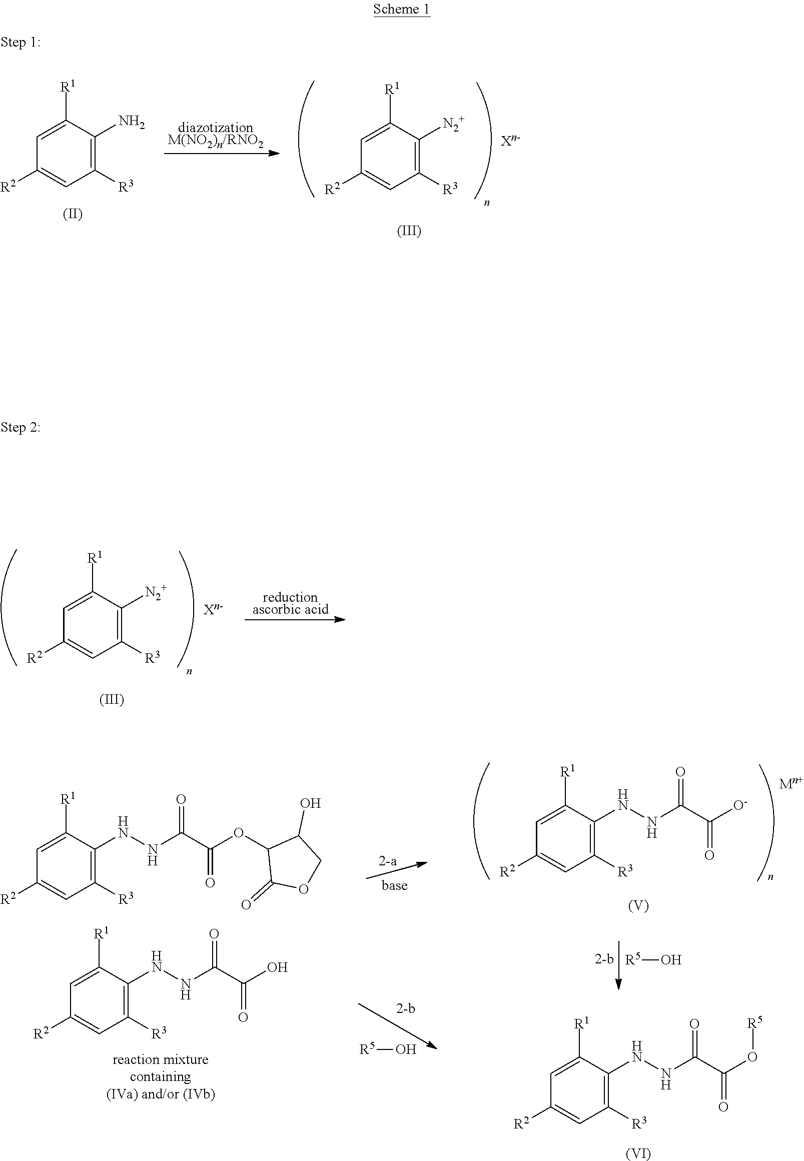

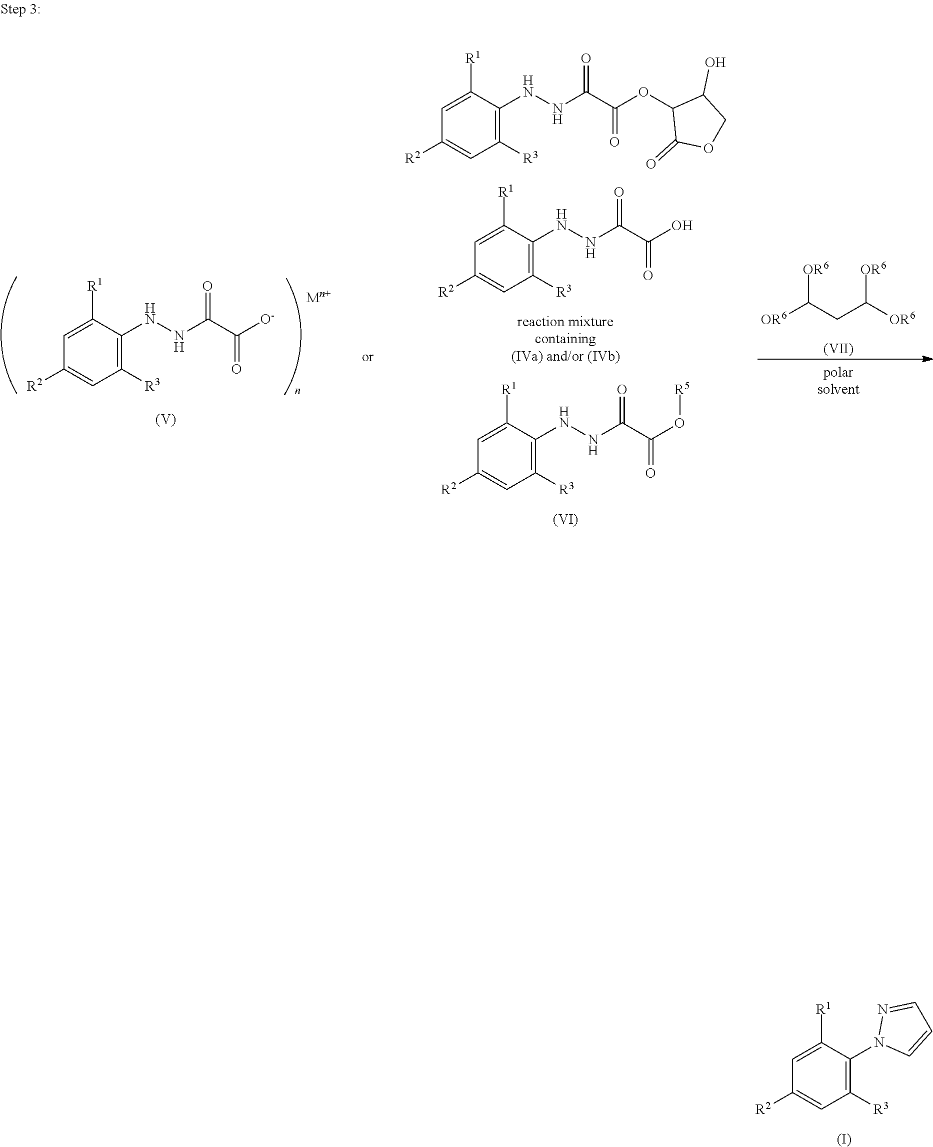

Scheme 1 gives a schematic overall representation of the process according to the invention, with all obligatory and optional steps. Reaction conditions and reactants are selected in this case in accordance with the above-described inventive and preferred configurations. All variables in the formulae (I), (II), (III), (IVa), (IVb), (V) and (VI) are defined as described above. In formula (VII), R⁶ in each case independently of each other are (C₁-C₄)-alkyl, preferably methyl or ethyl.

A preferred embodiment of the process according to the invention is as follows:

The compounds of the formula (II) are initially charged in an organic solvent and, after addition of an acid according to the invention, for example sulfuric acid, are admixed with sodium nitrite, for example dissolved in water, over 0.5 h to 3 h at preferably −10° C. to 80° C., particularly preferably 0° C. to 60° C. After addition is complete, ascorbic acid as reducing agent, for example as a solid or an aqueous solution, is added to the reaction mixture. After preferably 0.5 h to 6 h at −10° C. to 80° C., particularly preferably 0.5 h to 4 h at 0° C. to 60° C., a substance mixture containing the compounds of the formula (IVa) and/or (IVb) is isolated, for example after introducing the reaction mixture into water and subsequently filtering or extracting with an organic solvent. (Step (1) and (2))

Preferably, the isolated substance mixture containing the compounds (IVa) and (IVb) is subsequently admixed, in an organic solvent, for example methanol or ethanol, particularly preferably methanol, with addition of a strong acid, for example hydrochloric acid, sulfuric acid or methanesulfonic acid, particularly preferably sulfuric acid, with compounds of the general formula (VII), for example 1,1,3,3-tetramethoxypropane. Subsequently, the reaction mixture is preferably incubated with good stirring in a temperature range of 20° C. to 100° C., particularly preferably in a temperature range of 40° C. to 80° C., for a period of 2 to 15 hours until conversion is complete. (Step (3)) The compounds of the formula (I) formed can then be isolated and purified by the above-described methods.

A particularly preferred embodiment of the process according to the invention is as follows:

The compounds of the formula (II) are initially charged in acetic acid and, after addition of concentrated or aqueous sulfuric acid, are admixed with sodium nitrite over 0.5 h to 3 h at 0° C. to 60° C. After addition is complete, ascorbic acid as reducing agent, for example as a solid or an aqueous solution, is added to the reaction mixture. After preferably 0.5 h to 6 h at −10° C. to 80° C., particularly preferably 0.5 h to 4 h at 0° C. to 60° C., and complete conversion (HPLC^{a}), a substance mixture containing the compounds of the formula (IVa) and/or (IVb) is isolated, for example by introducing the reaction mixture into water and subsequently filtering or extracting with methyl tert-butyl ether. (Step (1) and (2)) Preferably, the isolated substance mixture containing the compounds (IVa) and/or (IVb) is subsequently admixed, in methanol after addition of concentrated sulfuric acid, with compounds of the general formula (VII), for example 1,1,3,3-tetramethoxypropane. Subsequently, the reaction mixture is preferably incubated with good stirring in a temperature range of 20° C. to 100° C., particularly preferably in a temperature range of 40° C. to 80° C., for a period of 2 to 15 hours until conversion is complete (HPLC^{a}). (Step (3)). The compounds of the formula (I) formed can then be isolated and purified by the above-described methods.

In one further preferred configuration of the process according to the invention, the preparation of the compounds of the formula (I) takes place over steps (1), (2), (2-a) and (3) or (1), (2), (2-a), (2-b) and (3). The isolated substance mixture containing the compounds of the general formula (IVa) and/or (IVb) after preparation thereof according to the invention, as described above for step (1) and (2), is preferably admixed, as a solution in an organic solvent, for example acetone, with an aqueous solution of a base, for example sodium hydroxide or sodium hydrogencarbonate. The reaction mixture is preferably incubated with good stirring in a temperature range of 10° C. to 35° C. for a period of 3 to 12 hours. (Step (2-a)) In one particularly preferred embodiment of the process according to the invention, the substance mixture containing the compounds of the general formula (IVa) and (IVb), after preparation thereof according to the invention as described above for step (1) and (2), is admixed, as a solution in acetone, with an aqueous solution of sodium hydrogencarbonate or sodium hydroxide or a mixture of these. The reaction mixture is preferably incubated with good stirring in a temperature range of 10° C. to 35° C. for a period of 3 to 12 hours. (Step (2-a))

The isolation of the compounds of the general formula (V) can take place, for example, by filtration, preferably with subsequent washing with water and optionally subsequent washing with an organic solvent.

Intermediates of the general formula (V) can be used directly in step (3) without any further workup. As an alternative, compounds of the formula (V) may be converted into compounds of the general formula (VI) in step (2-b) of the process according to the invention. Preferred configurations of step (2-b) are described below.

The compounds of the formula (V) or (VI) obtained can be further reacted in accordance with the above-described preferred configurations for step (3) to give compounds of the formula (I), which can then be isolated and purified according to the invention by the above-described methods.

In one further preferred configuration of the process according to the invention, the compounds of the formula (I) are prepared over steps (1), (2-b) and (3).

In one alternative preferred embodiment of the process according to the invention, the substance mixture containing the compounds of the general formula (IVa) and/or (IVb) is initially charged in an organic solvent of the formula R⁵—OH, for example methanol, and admixed with concentrated sulfuric acid. The reaction mixture is preferably incubated with good stirring in a temperature range of 30° C. to 90° C. for a period of 1 to 8 hours.

The thus-obtained intermediates of the general formula (VI) can be used directly in step (3) without any further workup. As an alternative, compounds of the formula (VI) can, by suitable workup steps generally known to those skilled in the art, be isolated and further characterized and subsequently be used in step (3).

The compounds of the formula (VI) obtained can be further reacted in accordance with the above-described preferred configurations for step (3) to give compounds of the formula (I), which can then be isolated and purified by the above-described methods.

In one further configuration of the process according to the invention, the compounds of the formula (I) are prepared over steps (1), (2) and (3), and optionally (2-b), in a one-pot reaction.

The term “one-pot reaction” is understood have to mean that the conversion of a compound of the formula (II) over steps (1), (2) and (3), and optionally (2-b), into a compound of the formula (I) meets at least one of the following conditions:
i) there is no isolation of the diazonium salt (III) from the reaction mixture of step (1);
ii) there is no purification of the diazonium salt (III) from the reaction mixture of step (1);
iii) there is no isolation of compounds of the formula (IVa), (IVb), (VI) or of any compounds of the formula (VIII) formed from the reaction mixture of step (2) or (2-b);

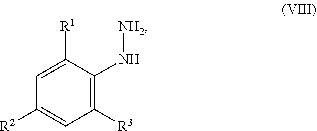

iv) there is no purification of compounds of the formula (IVa), (IVb), (VI) or of any compounds of the formula (VIII) formed from the reaction mixture of step (2) or (2-b);
v) all steps (1), (2), (3) and optionally (2-b) take place in the same reaction vessel;
vi) from the solvent of step (1) only a small proportion of the solvent is removed prior to the start of step (2) or prior to the start of step (2-b) or (3), preferably less than 50% by volume (percent by volume based on the volume of solvent used), preferably less than 30% by volume, more preferably less than 10% by volume, even more preferably at most 5% by volume of the solvent (e.g. by evaporation, for example at a reaction temperature of about 40° C., or active removal, e.g. by distillation and/or reduced pressure based on 1013 hPa), preferably no solvent is actively removed by the solvent exchange between step (1) and step (2), between step (2), any step (2-b), and (3) and, if present, between step (2) and (2-b) (e.g. by distillation and/or reduced pressure based on 1013 hPa);
vii) there is only a small exchange, preferably no exchange, of solvent between step (1) and (2) and between step (2) and (3) and, if present, between step (2) and (2-b) and between step (2-b) and (3), particularly preferably at most 50% by volume, preferably at most 40% by volume, more preferably at most 30% by volume, even more preferably at most 20% by volume of the solvent used in step 1 is replaced by a new solvent (the new solvent can be the same solvent or another solvent).

During the reaction sequence in a “one-pot” reaction, it is possible to add reaction volumes in the form of solids, liquids or suspensions, for example in the form of solid, dissolved or suspended reducing agents, or solvent (the same solvent as used prior to step (1) or another solvent), but with the aim of a reaction sequence without essential/without exchange of solvent as used in step (1) or active removal of solvent as used prior to step (1).

It is preferred in this configuration of the process according to the invention that neither the diazonium salt (III) formed after step (1) from compound (II) nor compounds of the formula (IVa), (IVb), (VI) or any compounds of the formula (VIII) formed are isolated or purified during the reaction sequence that leads to compound (I).

It is furthermore preferred in this configuration of the process according to the invention that neither the diazonium salt (III) formed after step (1) from compound (II) nor compounds of the formula (IVa), (IVb), (VI) or any compounds of the formula (VIII) formed are isolated or purified during the reaction sequence that leads to compound (I), nor is there any essential removal and/or exchange of solvent.

It is furthermore preferred in this configuration of the process according to the invention that neither the diazonium salt (III) formed after step (1) from compound (II) nor compounds of the formula (IVa), (IVb) (VI) or any compounds of the formula (VIII) formed are isolated or purified during the reaction sequence that leads to compound (I), nor is there any essential removal and/or exchange of solvent, and all of steps (1), (2) and (3) take place in the same reaction vessel. In this case, those skilled in the art will choose a reaction vessel from the start that can accommodate all volumes for reactions (1), (2) and (3).

In other words, it is preferable for the reaction sequence to be a telescoped reaction in one or more vessels, preferably one vessel.

In the context of the present invention, the term “purification” refers to the enrichment of a substance (and therefore depletion of other substances) to a purity of at least 20% by weight (percent by weight of a substance based on the total mass measured. The proportion may be determined chromatographically for example (e.g. HPLC or by gas chromatography or gravimetrically)), preferably at least 50% by weight, even more preferably at least 75% by weight, e.g. 90% by weight, 98% by weight or greater than 99% by weight.

The present invention moreover relates to the intermediate compounds of the formulae (IVa), (IVb), (V) and (VI).

The invention provides compounds of the formula (V)

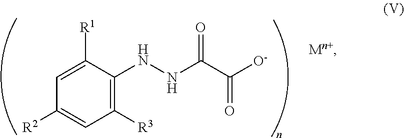

where R¹, R², R³ are as defined above, where R¹ and R³ are not simultaneously hydrogen in any compound, n is one or two and M is ammonium, an alkali metal, preferably Li, Na or K (with n=1), or an alkaline earth metal, preferably Mg, Ca or Ba (with n=2).

The invention further provides compounds of the formula (VI)

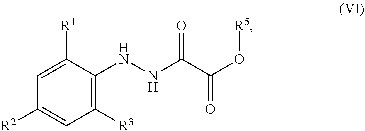

where R¹ and R³ are as defined above, where R¹ and R³ are not simultaneously hydrogen in any compound, R² is halogen-substituted C₁-C₄-alkyl or halogen-substituted C₁-C₄-alkoxy and R⁵ is C₁-C₄-alkyl, especially methyl or ethyl.

The invention further provides compounds of the formula (IVa) and (IVb)

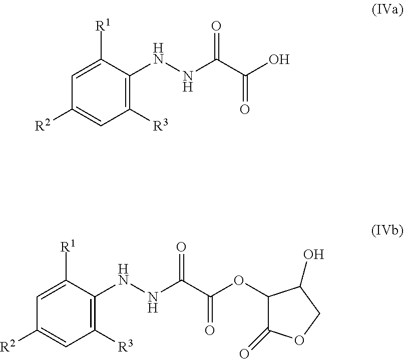

where R¹ and R³ are as defined above, where R¹ and R³ are not simultaneously hydrogen in any compound and R² is halogen-substituted C₁-C₄-alkyl or halogen-substituted C₁-C₄-alkoxy.

### EXAMPLES

The following examples explain the process according to the invention in more detail without limiting the invention thereto.

Methods:

The NMR data of the examples are listed in conventional form (6 values, multiplet splitting, number of hydrogen atoms).

The solvent and the frequency in which the NMR spectrum was recorded are stated in each case.

^{a}) HPLC (High Performance Liquid Chromatography) on a reversed-phase column (C18), Agilent 1100 LC system; Phenomenex Prodigy 100×4 mm ODS3; eluent A: acetonitrile (0.25 ml/1); eluent B: water (0.25 ml TFA/1); linear gradient from 5% acetonitrile to 95% acetonitrile in 7.00 min, then 95% acetonitrile for a further 1.00 min; oven temperature 40° C.; flow rate: 2.0 ml/min.

### 1) Step 1 and 2: Preparation of a Product Mixture Containing N-Arylhydrazino-2-Oxoacetic Acids (IVa)

### Example 1-1) 2-[2-[2,6-dichloro-4-[1,2,2,2-tetrafluoro-1-(trifluoromethyl)ethyl]phenyl]hydrazino]-2-oxoacetic acid (from Precursor of the General Formula (II)) (IVa-1)

25.0 g (74.5 mmol, 1.0 eq) of 2,6-dichloro-4-[1,2,2,2-tetrafluoro-1-(trifluoromethyl)ethyl]aniline were initially charged in 75 ml of acetonitrile and 75 ml of 50% by weight sulfuric acid and admixed at 0-5° C. with a solution of 5.65 g of sodium nitrite (82.0 mmol, 1.1 eq) in 10.0 ml of water over 30 min. After addition was complete, the reaction mixture was stirred for 15 min at this temperature and a solution of 14.4 g (82.0 mmol, 1.1 eq) of ascorbic acid in 50 ml of water was metered in over 1 h. After addition was complete, the reaction mixture was warmed to room temperature over 1.5 h. The reaction mixture was subsequently stirred for 5 h at 40° C. and, after cooling to room temperature and addition of 150 ml of water, the product was filtered and, after drying under reduced pressure at 40° C., obtained as a yellow-orange solid: yield 26.4 g (65% of theory)

¹H-NMR (CDCl₃, 400 MHz) δ (ppm)=9.21 (d, J=4.0 Hz, 1H), 7.54 (s, 2H), 6.82 (d, J=4.8 Hz, 1H).

### Example 1-2) 2-[2-[2,6-dichloro-4-[1,2,2,2-tetrafluoro-1-(trifluoromethyl)ethyl]phenyl]hydrazino]-2-oxoacetic acid (from Precursor of the General Formula (II)) (IVa-1)

53.4 g (136.0 mmol, 1.0 eq) of 2,6-dichloro-4-[1,2,2,2-tetrafluoro-1-(trifluoromethyl)ethyl]aniline were initially charged in 300 ml of glacial acetic acid and 150 ml of 50% by weight sulfuric acid and admixed at 0-5° C. with a solution of 11.3 g of sodium nitrite (163.0 mmol, 1.2 eq) in 20.0 ml of water over 30 min.

After addition was complete, the reaction mixture was stirred for 15 min at this temperature and a solution of 28.7 g (163.0 mmol, 1.2 eq) of ascorbic acid in 100 ml of water was metered in over 1 h. After addition was complete, the reaction mixture was warmed to room temperature over 1.5 h and washed with 200 ml of n-heptane. After addition of 500 ml of water, the product mixture was extracted with 500 ml of methyl tert-butyl ether, the organic phase was washed with 20% by weight NaCl solution and the crude product, after removal of the solvent under reduced pressure, was used directly in the next stage.

### Example 1-3) 2-[2-[2,6-dichloro-4-[1,2,2,2-tetrafluoro-1-(trifluoromethyl)ethyl]phenyl]hydrazino]-2-oxoacetic acid (from Precursor of the General Formula (II)) (IVa-1)

53.4 g (136.0 mmol, 1.0 eq) of 2,6-dichloro-4-[1,2,2,2-tetrafluoro-1-(trifluoromethyl)ethyl]aniline were initially charged in 300 ml of glacial acetic acid and 150 ml of 50% by weight sulfuric acid and admixed at 0-5° C. with a solution of 11.3 g of sodium nitrite (163.0 mmol, 1.2 eq) in 20.0 ml of water over 30 min.

After addition was complete, the reaction mixture was stirred for 15 min at this temperature and a solution of 28.7 g (163.0 mmol, 1.2 eq) of ascorbic acid in 100 ml of water was metered in over 1 h. After addition was complete, the reaction mixture was warmed to room temperature over 1.5 h and washed with 200 ml of n-heptane. After addition of 500 ml of water, the product mixture was filtered and the crude product, after drying under reduced pressure at 40° C., was used directly in the next stage.

### Step 2-a: Preparation of Sodium N-Arylhydrazino-2-Oxoacetate (V)

### Example 1-4) sodium 2-[2-[2,6-dichloro-4-[1,2,2,2-tetrafluoro-1-(trifluoromethyl)ethyl]phenyl]hydrazino]-2-oxoacetate (from Precursor Containing Compounds of the General Formula (IVa) and/or (IVb) from Step 2) (V-1)

2.84 g (68.0 mmol, 1.0 eq) of the 2,6-dichloro-4-[1,2,2,2-tetrafluoro-1-(trifluoromethyl)ethyl]phenyl]hydrazine derivative product mixture from step (2) were dissolved in 40 ml of acetone and admixed with 100 ml of water at room temperature. While monitoring the pH by means of a pH meter, the suspension is admixed dropwise with aqueous NaOH (10% by weight, approximately 37 ml) while stirring vigorously until a pH of 7.0 is reached. By adding 45.0 ml of saturated aqueous NaHCO₃ solution, the pH is adjusted to 7.5 and the suspension is stirred at this pH and room temperature for 12 h. After addition of a further 100 ml of water, the solid was filtered and the filter cake was washed with 200 ml of water and subsequently washed three times with in each case 50 ml of methyl tert-butyl ether. After drying under reduced pressure at 40° C., the product was obtained as a light beige solid: yield 11.6 g (78% of theory).

¹H-NMR (DMSO-d₆, 400 MHz) δ (ppm)=9.8 (br s, 1H), 7.70 (s, 1H), 7.49 (s, 2H).

### 2) Step 2-b: Preparation of the Alkyl N-Arylhydrazino-2-Oxoacetates (VI)

### Example 2-1) methyl 2-[2-[2,6-dichloro-4-[1,2,2,2-tetrafluoro-1-(trifluoromethyl)ethyl]phenyl]hydrazino]-2-oxoacetate (from Precursor of the General Formula (V) from step 2-a) (VI-1)

0.25 g (0.57 mmol, 1.0 eq) of sodium 2-[2-[2,6-dichloro-4-[1,2,2,2-tetrafluoro-1-(trifluoromethyl)ethyl]phenyl]hydrazino]-2-oxoacetate were dissolved in 2.5 ml of methanol and admixed dropwise with 0.06 g (0.57 mmol, 1.0 eq) of 96% by weight sulfuric acid at 0-5° C. After addition was complete, the solution was heated to 65° C. and stirred at this temperature for 3.5 h. After cooling to room temperature, the solution was stirred into 5 ml of water, the solid formed was filtered off and the product, after drying under reduced pressure at 40° C., was isolated as a colourless solid: yield 0.24 g (89% of theory).

¹H-NMR (CDCl₃, 400 MHz) δ (ppm)=9.05 (br d, J=5.0 Hz, 1H), 7.51 (s, 2H), 6.85 (d, J=5.0 Hz, 1H), 3.93 (s, 3H).

### Example 2-2) methyl 2-[2-[2,6-dichloro-4-[1,2,2,2-tetrafluoro-1-(trifluoromethyl)ethyl]phenyl]hydrazino]-2-oxoacetate (from Precursor Containing Compounds of the General Formula (IVa) and/or (IVb) from Step 2) (VI-1)

2.83 g (6.8 mmol, 1.0 eq) of the 2,6-dichloro-4-[1,2,2,2-tetrafluoro-1-(trifluoromethyl)ethyl]phenyl]hydrazine derivative product mixture from step (2) were dissolved in 15 ml of methanol and admixed dropwise with 0.74 g of 96% by weight sulfuric acid (6.8 mmol, 1.0 eq) at 0-5° C. After addition was complete, the solution was heated to 65° C. and stirred at this temperature for 3.5 h.

After cooling to room temperature, the solution was stirred into 50 ml of water, the solid formed was filtered off and the product, after drying under reduced pressure at 40° C., was isolated as a colourless solid: yield 2.3 g (80% of theory).

### 3) Step 3: Preparation of the N-Arylpyrazoles (I)

### Example 3-1) 1-[2,6-dichloro-4-(1,1,1,2,3,3,3-heptafluoropropan-2-yl)phenyl]-1H-pyrazole (from Precursor Containing Compounds of the General Formula (IVa) and/or (Vb) from Step 2) (I-1)

1.25 g (3.0 mmol, 1.0 eq) of the 2,6-dichloro-4-[1,2,2,2-tetrafluoro-1-(trifluoromethyl)ethyl]phenyl]hydrazine derivative product mixture from step (2) were initially charged in 2.5 ml of acetonitrile and 2.0 ml of water and admixed dropwise with 1.8 g of 50% by weight sulfuric acid (79.2 mmol, 3.0 eq) at 0-5° C. After addition was complete, the suspension was heated to 40° C., then admixed with 0.54 g (3.3 mmol, 1.1 eq) of 1,1,3,3-tetramethoxypropane and the reaction was stirred at 60° C. for 6 h. After cooling to room temperature, the mixture was extracted twice with 20 ml of n-heptane, the combined organic phases were washed with 20 ml of saturated sodium hydrogencarbonate solution and, after removal of the solvent under reduced pressure, the product was obtained as a yellow oil: yield: 0.5 g (30% of theory).

### Example 3-2) 1-[2,6-dichloro-4-(1,1,1,2,3,3,3-heptafluoropropan-2-yl)phenyl]-1H-pyrazole (from Precursor of the General Formula (V) from Step 2-a) (I-1)

11.6 g (26.4 mmol, 1.0 eq) of sodium 2-[2-[2,6-dichloro-4-[1,2,2,2-tetrafluoro-1-(trifluoromethyl)ethyl]phenyl]hydrazino]-2-oxoacetate were initially charged in 80 ml of methanol and admixed dropwise with 8.10 g (79.2 mmol, 3.0 eq) of 96% by weight sulfuric acid at 0-5° C. After addition was complete, the suspension was heated to 65° C. and, after stirring at this temperature for 1 h, admixed with 4.34 g (26.4 mmol, 1.0 eq) of 1,1,3,3-tetramethoxypropane. The reaction was stirred at this temperature for a further 7 h. After cooling to room temperature, after addition of 80 ml of water, the mixture was extracted once with 80 ml of n-heptane, and once more with 40 ml of n-heptane, the combined organic phases were washed with 80 ml of water and, after removal of the solvent under reduced pressure, the product was obtained as a yellow-orange oil: yield: 9.6 g (92% of theory).

¹H-NMR (CDCl₃, 400 MHz) δ (ppm)=7.85 (d, J=1.8 Hz, 1H), 7.71 (s, 2H), 7.61 (d, J=2.5 Hz, 1H), 6.55 (dd, J=1.8/2.5 Hz, 1H).

### Example 3-3) 1-[2,6-dichloro-4-(1,1,1,2,3,3,3-heptafluoropropan-2-yl)phenyl]-1H-pyrazole (from Precursor of the General Formula (VI) from Step 2-b) (I-1)

25.6 g (45%, 27.0 mmol, 1.0 eq) of methyl 2-[2-[2,6-dichloro-4-[1,2,2,2-tetrafluoro-1-(trifluoromethyl)ethyl]phenyl]hydrazino]-2-oxoacetate from step (2-b) were initially charged in 100 ml of acetonitrile and admixed dropwise with 1.3 g (13.5 mmol, 0.5 eq) of 96% by weight sulfuric acid and 1.7 g (54.0 mmol, 2.0 eq) of methanol. After addition was complete, 4.4 g (27.0 mmol, 1.0 eq) of 1,1,3,3-tetramethoxypropane were added and the reaction was heated to 60° C. The reaction was stirred at this temperature for 8 h. After cooling to room temperature, the solvent was removed under reduced pressure and the residue was separated between 150 ml of n-heptane and 100 ml of 10% by weight NaOH. The aqueous phase was extracted twice with 50 ml of n-heptane and the combined organic phases were washed with 100 ml of 10% by weight HCl and, after removal of the solvent under reduced pressure, the product was obtained as a dark yellow oil: yield: 10.1 g (90% of theory).

### 4) Preparation of the N-Arylpyrazoles (I), Step 2-b Together with Step 3

### Example 4-1) 1-[2,6-dichloro-4-(1,1,1,2,3,3,3-heptafluoropropan-2-yl)phenyl]-1H-pyrazole (from Precursor Containing Compounds of the General Formula (IVa) and/or (IVb) from Step 2) (I-1)

28.4 g (68.0 mmol, 1.0 eq) of the 2,6-dichloro-4-[1,2,2,2-tetrafluoro-1-(trifluoromethyl)ethyl]phenyl]hydrazine derivative product mixture from step (2) were initially charged in 150 ml of methanol and admixed dropwise with 13.89 g of 96% by weight sulfuric acid (136.0 mmol, 2.0 eq) at 0-5° C. After addition was complete, the solution was heated to 65° C. and, after stirring at this temperature for 0.5 h, admixed with 10.05 g (61.2 mmol, 0.9 eq) of 1,1,3,3-tetramethoxypropane. The reaction mixture was stirred at this temperature for a further 7 h. After cooling to room temperature, after addition of 100 ml of water, the mixture was extracted once with 100 ml of n-heptane and once more with 40 ml of n-heptane. The combined organic phases were washed with 150 ml of aqueous NaOH (10% by weight) and, after removal of the solvent under reduced pressure, the product was obtained as a yellow oil: yield: 21.8 g (80% of theory).

### Example 4-2) 1-[2,6-dichloro-4-(1,1,1,2,3,3,3-heptafluoropropan-2-yl)phenyl]-1H-pyrazole (from Precursor of the General Formula (II): One-Pot Method with Step 1, Step 2 and Step 3) (I-1)

27.9 g (68.0 mmol, 1.0 eq) of 2,6-dichloro-4-[1,2,2,2-tetrafluoro-1-(trifluoromethyl)ethyl]aniline were initially charged in 150 ml of glacial acetic acid and 75 ml of sulfuric acid (50% by weight) and admixed with a solution of 5.4 g of sodium nitrite (78.2 mmol, 1.15 eq) in 10.0 ml of water over 30 min at 0-5° C. After addition was complete, the reaction mixture was stirred for 15 min at this temperature and then 14.0 g (78.2 mmol, 1.15 eq) of ascorbic acid were added in one portion. The reaction mixture was warmed to room temperature over 2 h, then heated to 65° C., and 11.3 g (68.0 mmol, 1.0 eq) of 1,1,3,3-tetramethoxypropane were added at this temperature. The reaction was stirred at this temperature for a further 5 h. After cooling to room temperature and addition of 250 ml of water, the mixture was extracted once with 200 ml of n-heptane and once with 100 ml of n-heptane, the combined organic phases were washed with 150 ml of 10% by weight aqueous NaOH and the product was obtained, after removal of the solvent under reduced pressure, as an orange-red oil: yield 22.6 g (85% of theory).

The following N-arylpyrazoles of the general formula (I) were preparable analogously to example (4-1):

### 1-[2-Bromo-4-(1,1,1,2,3,3,3-heptafluoropropan-2-yl)-6-(trifluoromethoxy)phenyl]-1H-pyrazole (I-2)

¹H-NMR (CDCl₃, 400 MHz) δ (ppm)=7.92 (d, J=1.8 Hz, 1H), 7.84 (d, J=1.8 Hz, 1H), 7.62 (s, 1H), 7.61 (d, J=2.5 Hz, 1H), 6.54 (dd, J=1.8/2.5 Hz, 1H).

### 1-[2-Chloro-4-(1,1,1,2,3,3,3-heptafluoropropan-2-yl)-6-(trifluoromethoxy)phenyl]-1H-pyrazole (I-3)

¹H-NMR (CDCl₃, 400 MHz) δ (ppm)=7.85 (d, J=1.9 Hz, 1H), 7.76 (d, J=1.9 Hz, 1H), 7.62 (d, J=2.5 Hz, 1H), 7.59 (s, 1H), 6.54 (dd, J=1.9/2.5 Hz, 1H).

### 1-[2-Chloro-4-[1,2,2,2-tetrafluoro-1-(trifluoromethyl)ethyl]-6-(trifluoromethyl)phenyl]-1H-pyrazole (I-4)

¹H-NMR (DMSO-d₆, 400 MHz) δ (ppm)=8.43 (br s, 1H), 8.14 (d, J=2.5 Hz, 1H), 8.03 (br s, 1H), 7.86 (d, J=1.8 Hz, 1H), 6.69 (dd, J=1.8/2.5 Hz, 1H).

### 1-[2-Bromo-6-chloro-4-[1,2,2,2-tetrafluoro-1-(trifluoromethyl)ethyl]phenyl]-1H-pyrazole (I-5)

¹H-NMR (DMSO-d₆, 400 MHz) δ (ppm)=8.12 (dd, J=0.6/2.5 Hz, 1H), 8.10 (br d, J=1.8 Hz, 1H), 8.06 (br d, J=1.8 Hz, 1H), 7.84 (dd, J=0.6/2.5 Hz, 1H), 6.59 (dd, J=1.8/2.5 Hz, 1H).

### 1-[2-Bromo-4-[1,2,2,2-tetrafluoro-1-(trifluoromethyl)ethyl]-6-(trifluoromethyl)phenyl]-1H-pyrazole (I-6)

¹H-NMR (DMSO-d₆, 400 MHz) δ (ppm)=8.50 (br s, 1H), 8.13 (d, J=2.5 Hz, 1H), 8.06 (br s, 1H), 7.84 (dd, J=1.8/2.5 Hz, 1H), 6.59 (dd, J=1.8/2.5 Hz, 1H).

### 1-[2-Methyl-4-[1,2,2,2-tetrafluoro-1-(trifluoromethyl)ethyl]-6-(trifluoromethyl)phenyl]-1H-pyrazole (I-7)

¹H-NMR (CDCl₃, 400 MHz) δ (ppm)=7.87 (br s, 1H), 7.80 (d, J=1.8 Hz, 1H), 7.77 (br s, 1H), 7.56 (dd, J=0.7/1.8 Hz, 1H), 6.52 (dd, J=0.7/1.8 Hz, 1H), 2.09 (s, 3H).

The following intermediates of the general formula (IVa) were preparable analogously to example (4-1):

### 2-[2-[2-Chloro-4-[1,2,2,2-tetrafluoro-1-(trifluoromethyl)ethyl]-6-(trifluoromethoxy)phenyl]hydrazino]-2-oxoacetic acid (IVa-2)

¹H-NMR (DMSO-d₆, 400 MHz) δ (ppm)=14.2 (br s, 1H), 11.04 (s, 1H), 8.42 (s, 1H), 7.63 (d, J=2.0 Hz, 1H), 7.39 (s, 1H).

### 2-[2-[2-Bromo-4-[1,2,2,2-tetrafluoro-1-(trifluoromethyl)ethyl]-6-(trifluoromethoxy)phenyl]hydrazino]-2-oxoacetic acid (IVa-3)

¹H-NMR (DMSO-d₆, 400 MHz) δ (ppm)=14.1 (br s, 1H), 11.04 (s, 1H), 8.23 (s, 1H), 7.74 (d, J=2.0 Hz, 1H), 7.42 (s, 1H).

### 2-[2-[2-Chloro-4-[1,2,2,2-tetrafluoro-1-(trifluoromethyl)ethyl]-6-(trifluoromethyl)phenyl]hydrazino]-2-oxoacetic acid (IVa-4)

¹H-NMR (DMSO-d₆, 400 MHz) δ (ppm)=8.07 (br s, 1H), 7.84 (d, J=1.9 Hz, 1H), 7.58 (d, J=1.6 Hz, 1H).

### 2-[2-[2-Methyl-4-[1,2,2,2-tetrafluoro-1-(trifluoromethyl)ethyl]-6-(trifluoromethyl)phenyl]hydrazino]-2-carboxylic acid (IVa-5)

¹H-NMR (DMSO-d₆, 400 MHz) δ (ppm)=10.84 (s, 1H), 7.75 (s, 1H), 7.60 (s, 1H), 7.51 (s, 1H).

### 2-[2-[2-Bromo-4-[1,2,2,2-tetrafluoro-1-(trifluoromethyl)ethyl]-6-(trifluoromethyl)phenyl]hydrazino]-2-oxoacetic acid (IVa-6)

¹H-NMR (DMSO-d₆, 400 MHz) δ (ppm)=11.0 (s, 1H), 8.13 (s, 1H), 8.01 (s, 1H), 7.66 (s, 1H).

The following intermediates of the general formula (V) were preparable analogously to example (1-4):

### Sodium 2-[2-[2-chloro-4-[1,2,2,2-tetrafluoro-1-(trifluoromethyl)ethyl]-6-(trifluoromethoxy)phenyl]hydrazino]-2-oxoacetate (V-2)

¹H-NMR (DMSO-d₆, 400 MHz) δ (ppm)=9.92 (br s, 1H), 8.10 (s, 1H), 7.56 (s, 1H), 7.34 (s, 1H).

### Sodium 2-[2-[2-bromo-4-[1,2,2,2-tetrafluoro-1-(trifluoromethyl)ethyl]-6-(trifluoromethoxy)phenyl]hydrazino]-2-oxoacetate (V-3)

¹H-NMR (DMSO-d₆, 400 MHz) δ (ppm)=9.90 (br s, 1H), 7.88 (s, 1H), 7.69 (s, 1H), 7.38 (s, 1H).

### Sodium 2-[2-[2-chloro-4-[1,2,2,2-tetrafluoro-1-(trifluoromethyl)ethyl]-6-(trifluoromethyl)phenyl]hydrazino]-2-oxoacetate (V-4)

¹H-NMR (DMSO-d₆, 400 MHz) δ (ppm)=11.00 (s, 1H), 8.38 (s, 1H), 7.90 (s, 1H), 7.62 (s, 1H).

### Sodium 2-[2-[2-methyl-4-[1,2,2,2-tetrafluoro-1-(trifluoromethyl)ethyl]-6-(trifluoromethyl)phenyl]hydrazino]-2-oxoacetate (V-5)

¹H-NMR (DMSO-d₆, 400 MHz) δ (ppm)=7.53 (s, 1H), 7.47 (s, 1H), 7.41 (s, 1H).

### Sodium 2-[2-[2-bromo-4-[1,2,2,2-tetrafluoro-1-(trifluoromethyl)ethyl]-6-(trifluoromethyl)phenyl]hydrazino]-2-oxoacetate (V-6)

¹H-NMR (DMSO-d₆, 400 MHz) δ (ppm)=7.97 (s, 1H), 7.84 (s, 1H), 7.63 (s, 1H).

The following intermediates of the general formula (VI) were preparable analogously to examples (2-1) and (2-2):

### Ethyl 2-[2-[2,6-dichloro-4-[1,2,2,2-tetrafluoro-1-(trifluoromethyl)ethyl]phenyl]hydrazino]-2-oxoacetate (VI-2)

¹H-NMR (DMSO-d₆, 400 MHz) δ (ppm)=11.15 (d, J=1.1 Hz, 1H), 8.12 (d, J=1.1 Hz, 1H), 7.56 (s, 2H), 4.27 (q, J=7.1 Hz, 2H), 1.27 (t, J=7.1 Hz, 3H).

### Isopropyl 2-[2-[2,6-dichloro-4-[1,2,2,2-tetrafluoro-1-(trifluoromethyl)ethyl]phenyl]hydrazino]-2-oxoacetate (VI-3)

¹H-NMR (CDCl₃, 400 MHz) δ (ppm)=11.11 (br s, 1H), 8.11 (br s, 1H), 7.56 (s, 1H), 5.05 (sept., J=6.2 Hz, 1H), 1.27 (d, J=6.2 Hz, 6H).

### Methyl 2-[2-[2-chloro-4-[1,2,2,2-tetrafluoro-1-(trifluoromethyl)ethyl]-6-(trifluoromethoxy)phenyl]hydrazino]-2-oxoacetate (VI-4)

¹H-NMR (CDCl₃, 400 MHz) δ (ppm)=8.95 (d, J=4.6 Hz, 1H), 7.54 (d, J=1.9 Hz, 1H), 7.38 (s, 1H), 6.75 (d, J=4.6 Hz, 1H), 3.94 (s, 3H).

### Ethyl 2-[2-[2-chloro-4-[1,2,2,2-tetrafluoro-1-(trifluoromethyl)ethyl]-6-(trifluoromethoxy)phenyl]hydrazino]-2-oxoacetate (VI-5)

¹H-NMR (CDCl₃, 400 MHz) δ (ppm)=8.98 (br s, 1H), 7.54 (s, 1H), 7.38 (s, 1H), 6.75 (s, 1H), 4.37 (q, J=7.2 Hz, 2H), 1.38 (t, J=7.2 Hz, 3H).

### Methyl 2-[2-[2-bromo-4-[1,2,2,2-tetrafluoro-1-(trifluoromethyl)ethyl]-6-(trifluoromethoxy)phenyl]hydrazino]-2-oxoacetate (VI-6)

¹H-NMR (CDCl₃, 400 MHz) δ (ppm)=8.99 (d, J=4.6 Hz, 1H), 7.65 (d, J=1.9 Hz, 1H), 7.42 (s, 1H), 6.75 (d, J=4.6 Hz, 1H), 3.94 (s, 3H).

### Ethyl 2-[2-[2-bromo-4-[1,2,2,2-tetrafluoro-1-(trifluoromethyl)ethyl]-6-(trifluoromethoxy)phenyl]hydrazino]-2-oxoacetate (VI-7)

¹H-NMR (CDCl₃, 400 MHz) δ (ppm)=8.98 (d, J=4.6 Hz, 1H), 7.69 (d, J=1.9 Hz, 1H), 7.42 (s, 1H), 6.75 (d, J=4.6 Hz, 1H), 4.37 (q, J=7.2 Hz, 2H), 1.37 (t, J=7.2 Hz, 3H).

### Methyl 2-[2-[2-chloro-4-[1,2,2,2-tetrafluoro-1-(trifluoromethyl)ethyl]-6-(trifluoromethyl)phenyl]hydrazino]-2-oxoacetate (as 1:1 mixture of rotamers) (VI-8)

¹H-NMR (DMSO-d₆, 400 MHz) δ (ppm)=11.21 (s, 1H), 8.43 (s, 1H), 7.90 (d, J=1.9 Hz, 1H), 7.70 (d, J=1.9 Hz, 1H), 7.62 (d, J=1.9 Hz, 1H), 7.58 (d, J=1.9 Hz, 1H), 3.82 (s, 3H).

### Ethyl 2-[2-[2-chloro-4-[1,2,2,2-tetrafluoro-1-(trifluoromethyl)ethyl]-6-(trifluoromethyl)phenyl]hydrazino]-2-oxoacetate (VI-9)

¹H-NMR (DMSO-d₆, 400 MHz) δ (ppm)=11.12 (s, 1H), 8.42 (s, 1H), 7.91 (d, J=1.9 Hz, 1H), 7.63 (d, J=1.6 Hz, 1H), 7.15 (q, J=7.2 Hz, 2H), 1.27 (t, J=7.2 Hz, 3H).

### Methyl 2-[2-[2-bromo-4-[1,2,2,2-tetrafluoro-1-(trifluoromethyl)ethyl]-6-(trifluoromethyl)phenyl]hydrazino]-2-oxoacetate (VI-10)

¹H-NMR (DMSO-d₆, 400 MHz) δ (ppm)=11.02 (s, 1H) 8.18 (s, 1H), 8.02 (s, 1H), 7.66 (s, 1H), 3.82 (s, 3H).

### Methyl 2-[2-[2-bromo-6-chloro-4-[1,2,2,2-tetrafluoro-1-(trifluoromethyl)ethyl]phenyl]hydrazino]-2-oxoacetate (VI-11)

¹H-NMR (DMSO-d₆, 400 MHz) δ (ppm)=11.12 (s, 1H) 7.88 (s, 1H), 7.68 (s, 1H), 7.59 (s, 1H), 3.82 (s, 3H).

### Methyl 2-[2-[2-methyl-4-[1,2,2,2-tetrafluoro-1-(trifluoromethyl)ethyl]-6-(trifluoromethyl)phenyl]hydrazino]-2-oxoacetate (VI-12)

¹H-NMR (CDCl₃, 400 MHz) δ (ppm)=8.74 (br s, 1H), 7.68 (s, 1H), 7.55 (s, 1H), 6.27 (br s, 1H), 3.93 (s, 3H).

### Ethyl 2-[2-[2-methyl-4-[1,2,2,2-tetrafluoro-1-(trifluoromethyl)ethyl]-6-(trifluoromethyl)phenyl]hydrazino]-2-oxoacetate (VI-13)

¹H-NMR (CDCl₃, 400 MHz) δ (ppm)=8.75 (br d, J=3.0 Hz, 1H), 7.68 (s, 1H), 7.52 (s, 1H), 6.27 (br d, J=3.0 Hz, 1H), 4.38 (q, J=7.2 Hz, 2H), 1.38 (t, J=7.2 Hz, 3H).

### Ethyl 2-[2-[2-bromo-4-[1,2,2,2-tetrafluoro-1-(trifluoromethyl)ethyl]-6-(trifluoromethyl)phenyl]hydrazino]-2-oxoacetate (VI-14)

¹H-NMR (DMSO-d₆, 400 MHz) δ (ppm)=7.95 (s, 1H), 7.68 (s, 1H), 7.42 (br s, 1H), 4.27 (q, J=7.1 Hz, 2H), 1.25 (t, J=7.1 Hz, 3H).

### Comparative Examples with Respect to the Adverse Effect of Water in the Case of Small Amounts of Acid

### 2-[2-[2,6-Dichloro-4-[1,2,2,2-tetrafluoro-1-(trifluoromethyl)ethyl]phenyl]hydrazino]-2-oxoacetic acid (from Precursor of the General Formula (II)) (IVa-1)

6.2 g (13.6 mmol, 1.0 eq) of 2,6-dichloro-4-[1,2,2,2-tetrafluoro-1-(trifluoromethyl)ethyl]aniline were initially charged in 30 ml of acetonitrile and 30 ml of 10% by weight sulfuric acid and admixed at 0-5° C. with a solution of 1.3 g of sodium nitrite (16.3 mmol, 1.2 eq) in 2.0 ml of water over 15 min. After addition was complete, the reaction mixture was stirred for 15 min at this temperature and a solution of 2.8 g (16.3 mmol, 1.2 eq) of ascorbic acid in 10 ml of water was metered in over 1 h. After addition was complete, the reaction mixture was heated to room temperature over 1.5 h. 37% of unreacted starting material was still detected by means of HPLC^{a}, as was the formation of approximately 30% of undesired secondary components. The product was not isolated.

### 2-[2-[2,6-Dichloro-4-[1,2,2,2-tetrafluoro-1-(trifluoromethyl)ethyl]phenyl]hydrazino]-2-oxoacetic acid (from Precursor of the General Formula (II)) (IVa-1)

8.0 g (17.2 mmol, 1.0 eq) of 2,6-dichloro-4-[1,2,2,2-tetrafluoro-1-(trifluoromethyl)ethyl]aniline were initially charged in 20 ml of acetonitrile and 8.0 g (41.3 mmol, 2.4 eq) of 50% by weight sulfuric acid and admixed at 0-5° C. with a solution of 1.4 g of sodium nitrite (19.7 mmol, 1.15 eq) in 2.5 ml of water over 15 min. After addition was complete, the reaction mixture was stirred for 15 min at this temperature and then 3.8 g (21.5 mmol, 1.25 eq) of ascorbic acid were added. After addition was complete, the reaction mixture was warmed to room temperature over 1.5 h. 17% of unreacted starting material was still detected by means of HPLC^{a}, as was the formation of approximately 8% of secondary components. The product was not isolated.

### Preparation of the Precursors of the Formula (II)

### 4-[1,2,2,2-Tetrafluoro-1-(trifluoromethyl)ethyl]aniline

60.0 g (0.64 mol, 1.0 eq) of aniline were initially charged in 450 ml each of water and ethyl acetate and admixed successively with 4.5 g (13.0 mmol, 0.02 eq) of tetra-n-butylammonium hydrogensulfate and 144.0 g (0.70 mol, 1.1 eq) of sodium dithionite. 214.0 g (0.70 mol, 1.1 eq) of heptafluoroisopropyl iodide were metered in at room temperature over 3 h and the pH was maintained at 6.0-7.0 during the metering by adding 40% by weight aqueous K₂CO₃. After addition was complete, stirring was carried out for a further 3 h at approximately 21° C., then the phases were separated and the organic phase was washed with a solution of 40 ml each of 20% by weight NaCl and 2.5% by weight HCl. After removal of the solvent under reduced pressure, the product was obtained as a reddish oil: yield: 180.0 g (98% of theory).

¹H-NMR (CDCl₃, 400 MHz) δ (ppm)=7.35 (d, J=8.9 Hz, 2H), 6.72 (d, J=7.7 Hz, 2H), 3.91 (br s, 2H).

### 2,6-Dichloro-4-[1,2,2,2-tetrafluoro-1-(trifluoromethyl)ethyl]aniline

180.0 g (0.64 mmol, 1.0 eq) of 4-[1,2,2,2-tetrafluoro-1-(trifluoromethyl)ethyl]aniline were initially charged in 600 ml of ethyl acetate and 100 ml of water and admixed with 96.0 g (128.0 mmol, 2.0 eq) of chlorine gas over 5 h at 0-5° C. The phases were subsequently separated and the aqueous phase was extracted successively with a mixture of 100 ml of ethyl acetate and 50 ml of n-heptane and also a mixture of 50 ml of ethyl acetate and 25 ml of n-heptane. The combined organic phases were washed twice with 100 ml each time of 20% by weight NaCl solution and the product, after removal of the solvent, was obtained as a red-brown oil: yield 200.0 g (95% of theory).

¹H-NMR (CDCl₃, 400 MHz) δ (ppm)=7.41 (s, 2H), 4.76 (br s, 2H).

### 4-[1,2,2,2-Tetrafluoro-1-(trifluoromethyl)ethyl]-2-(trifluoromethoxy)aniline

40.0 g (0.22 mol, 1.0 eq) of 2-trifluoromethoxyaniline were initially charged in 400 ml of water and 250 ml of ethyl acetate and admixed successively with 1.55 g (4.4 mmol, 0.02 eq) of tetra-n-butylammonium hydrogensulfate and 68.0 g (0.33 mol, 1.5 eq) of sodium dithionite. 100.2 g (0.33 mol, 1.5 eq) of heptafluoroisopropyl iodide were metered in at room temperature over 2.5 h and the pH was maintained at 4.0-5.0 during the metering by adding 40% by weight aqueous K₂CO₃. After addition was complete, stirring was carried out for a further 1 h at approximately 21° C., then the phases were separated. The organic phase was diluted with 100 ml of n-heptane, then washed with 250 ml of 20% by weight HCl, with 250 ml of saturated NaCl solution and with 250 ml of water. After removal of the solvent under reduced pressure, the product was obtained as a yellow oil: yield 76.4 g (92% of theory).

¹H-NMR (CDCl₃, 400 MHz) δ (ppm)=7.36 (s, 1H), 7.30 (d, J=8.6 Hz, 1H), 6.85 (d, J=8.6 Hz, 1H), 4.18 (br s, 2H).

### 2-Chloro-4-[1,2,2,2-tetrafluoro-1-(trifluoromethyl)ethyl]-6-(trifluoromethoxy)aniline

30.0 g (79.7 mmol, 1.0 eq) of 4-[1,2,2,2-tetrafluoro-1-(trifluoromethyl)ethyl]-2-(trifluoromethoxy)aniline were dissolved in 120 ml of DMF and heated to 80° C. 14.2 g (79.7 mmol, 1.0 eq) of N-chlorosuccinimide were added in portions over 2 h at this temperature. After addition was complete, further stirring was carried out for 30 min at this temperature and, after cooling to room temperature, the mixture was separated between 200 ml of water and 100 ml of n-heptane and the organic phase was subsequently washed with 100 ml of water. After removal of the solvent under reduced pressure, the product was obtained as a brown oil: yield 30.3 g (99% of theory)

¹H-NMR (CDCl₃, 400 MHz) δ (ppm)=7.45 (s, 1H), 7.30 (s, 1H), 4.59 (s, 2H).

### 2-Bromo-4-[1,2,2,2-tetrafluoro-1-(trifluoromethyl)ethyl]-6-(trifluoromethoxy)aniline

30.0 g (79.7 mmol, 1.0 eq) of 4-[1,2,2,2-tetrafluoro-1-(trifluoromethyl)ethyl]-2-(trifluoromethoxy)aniline were dissolved in 120 ml of DMF and heated to 80° C. 10.9 g (79.7 mmol, 1.0 eq) of N-bromosuccinimide were added in portions over 2 h at this temperature. After addition was complete, further stirring was carried out for 30 min at this temperature and, after cooling to room temperature, the mixture was separated between 200 ml of water and 100 ml of n-heptane and the organic phase was subsequently washed with 100 ml of water. After removal of the solvent under reduced pressure, the product was obtained as a brown oil: yield 31.6 g (93% of theory)

¹H-NMR (CDCl₃, 400 MHz) δ (ppm)=7.59 (s, 1H), 7.34 (s, 1H), 4.65 (br s, 2H).

1: A process for preparing a compound of formula (I) 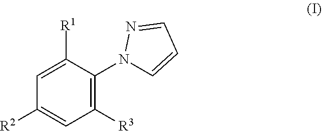
wherein
R¹ is hydrogen, cyano, halogen, C₁-C₄-alkyl optionally substituted by halogen or CN, or C₁-C₄-alkoxy optionally substituted by halogen,
R² is trifluoromethylsulfonyl, trifluoromethylsulfinyl, trifluoromethylsulfanyl, halogen, C₁-C₄-alkyl optionally substituted by halogen, or C₁-C₄-alkoxy optionally substituted by halogen, and
R³ is hydrogen, cyano, halogen, C₁-C₄-alkyl optionally substituted by halogen or CN, or C₁-C₄-alkoxy optionally substituted by halogen,
where R¹ and R³ are not simultaneously hydrogen in any compound,
starting from a compound of formula (II) wherein R¹, R² and R³ are as defined for formula (I) and wherein R¹ and R³ are not simultaneously hydrogen in any compound, 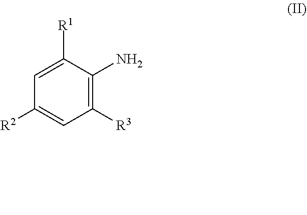
comprising the following steps (1) to (3)
(1) diazotization with of the compound of formula (II) with a compound of formula RNO₂ or M(NO₂)ₙ, wherein R is (C₁-C₆)-alkyl, n is one or two and M is ammonium, an alkali metal (with n=1) or an alkaline earth metal (with n=2), and at least one acid selected from mineral acids, sulfonic acids or carboxylic acids, wherein the carboxylic acids have a pKa of ≤2,
(2) reduction with ascorbic acid; and
(3) cyclization with a 1,1,3,3-tetra(C₁-C₄)alkoxypropane in a polar solvent in the presence of at least one acid selected from mineral acids, sulfonic acids or carboxylic acids, where the carboxylic acids have a pKa ≤2.

2: The process according to claim 1, wherein, after step (2), a base is added in a further step (2-a) and compounds of the formula (V) are precipitated out as a result 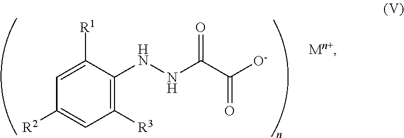
where R¹, R², R³ are defined according to claim 1, where R¹ and R³ are not simultaneously hydrogen in any compound, n is one or two and M is ammonium, an alkali metal (with n=1) or an alkaline earth metal (with n=2).

3: The process according to claim 1, wherein, after step (2), or step (2-a), in a further step (2-b), at least one compound of the formula R⁵—OH is added, as a result of which, in the presence of at least one acid selected from mineral acids or sulfonic acids, compounds of the formula (VI) are formed, 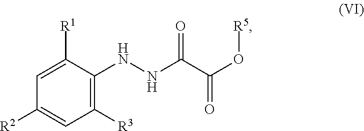
where R¹, R², R³ are defined according to claim 1, where R¹ and R³ are not simultaneously hydrogen in any compound and R⁵ is C₁-C₄-alkyl.

4: The process according to claim 1, wherein, after step (1), diazonium salts of the formula (III) are formed and these are then further reacted in step (2), 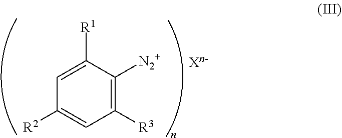
where R¹, R², R³ are defined according to claim 1, where R¹ and R³ are not simultaneously hydrogen in any compound and Xⁿ⁻ is a corresponding base of the acids according to claim 1, step (1), and n is 1 or 2.

5: The process according to claim 1, wherein, after step (2), a reaction mixture comprising intermediate compounds of the formula (IVa) and/or (IVb) is formed and this is then further reacted in step (3), (2-a) or (2-b) 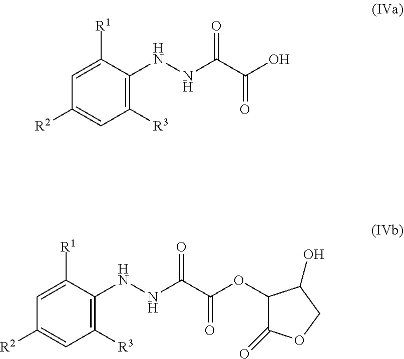
where R¹, R², R³ are defined according to claim 1, where R¹ and R³ are not simultaneously hydrogen in any compound.

6: The process according to claim 1, wherein R² is halogen-substituted C₁-C₄-alkyl or halogen-substituted C₁-C₄-alkoxy.

7: The process according to claim 1, wherein R¹ and R³ in each case independently of one another are a substituent selected from hydrogen, Cl, Br, F, C₁-C₃-alkyl, halogen-substituted C₁-C₃-alkyl, C₁-C₃-alkoxy or halogen-substituted C₁-C₃-alkoxy.

8: The process according to claim 1, wherein
R¹ is halogen or (C₁-C₃)-alkyl,
R² is fluorine-substituted C₁-C₄-alkyl or fluorine-substituted C₁-C₄-alkoxy and
R³ is halogen, C₁-C₃-alkyl or fluorine-substituted C₁-C₃-alkyl, C₁-C₃-alkoxy or fluorine-substituted C₁-C₃-alkoxy.

9: The process according to claim 2, wherein the base in step (2-a) is selected from hydrogencarbonates, in particular NaHCO₃ or KHCO₃, carbonates, in particular Na₂CO₃ or K₂CO₃, or hydroxides, in particular NaOH or KOH.

10: The process according to claim 1, wherein the acid in step (1) is used in pure form or as an aqueous solution at concentrations from 10-99% by weight.

11: The process according to claim 3, wherein the alcohol R⁵—OH in step (2-b) is used simultaneously as solvent and reagent.

12: The process according to claim 3, wherein the compound R⁵—OH from step (2-b) is used as solvent for step (2-b) and step (3).

13: The process according to claim 1, wherein the said process comprises or consists of the steps (1), (2), (2-a), (2-b) and (3).

14: The process according to claim 1, wherein the said process comprises or consists of the steps (1), (2), (2-b) and (3).

15: The process according to claim 1, wherein the steps (1) and (2) are carried out together in a “one-pot” reaction, wherein the diazonium salt (III) formed after step (1) from compound (II) is not isolated or purified.

16: The process according to claim 3, wherein the steps (2-b) and (3) are carried out together in a “one-pot” reaction, wherein the compound (VI) formed after step (2-b) is not isolated or purified.

17: The process according to claim 1, wherein the said process is carried out as a “one-pot” reaction.

18: The process according to claim 17, wherein the conversion of a compound of the formula (II) over steps (1), (2) and (3), and optionally (2-b), into a compound of the formula (I) meets at least one of the following conditions:
i) there is no isolation of the diazonium salt (III) from the reaction mixture of step (1);
ii) there is no purification of the diazonium salt (III) from the reaction mixture of step (1);
iii) there is no isolation of compounds of the formula (IVa), (IVb), (VI) or of any compounds of the formula (VIII) formed from the reaction mixture of step (2) or (2-b); 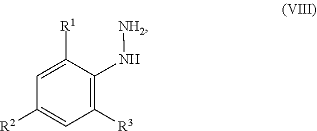
iv) there is no purification of compounds of the formula (IVa), (IVb), (VI) or of any compounds of the formula (VIII) formed from the reaction mixture of step (2) or (2-b);
v) all steps (1), (2) and (3) and optionally (2-b) take place in the same reaction vessel;
vi) from the solvent of step (1) only a small proportion of the solvent is removed prior to the start of step (2) or prior to the start of step (2-b) or (3), preferably less than 50% by volume (percent by volume based on the volume of solvent used), preferably less than 30% by volume, more preferably less than 10% by volume, even more preferably at most 5% by volume of the solvent (e.g. by evaporation, for example at a reaction temperature of about 40° C., or active removal, e.g. by distillation and/or reduced pressure based on 1013 hPa), preferably no solvent is actively removed by the solvent exchange between step (1) and step (2), between step (2), any step (2-b), and (3) and, if present, between step (2) and (2-b) (e.g. by distillation and/or reduced pressure based on 1013 hPa);
vii) there is only a small exchange, preferably no exchange, of solvent between step (1) and (2) and between step (2) and (3) and, if present, between step (2) and (2-b) and between step (2-b) and (3), particularly preferably at most 50% by volume, preferably at most 40% by volume, more preferably at most 30% by volume, even more preferably at most 20% by volume, of the solvent used in step 1 is replaced by a new solvent (the new solvent can be the same solvent or another solvent).

19: The process according to claim 17, wherein neither the diazonium salt (III) formed after step (1) from compound (II) nor compounds of the formula (IVa), (IVb), (VI) or any compounds of the formula (VIII) formed are isolated or purified during the reaction sequence that leads to compound (I).

20: A compound of formula (V) 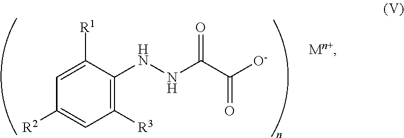
where R¹, R², R³ are defined according to claim 1,
wherein R¹ and R³ are not simultaneously hydrogen in any compound, n is one or two and M is ammonium, an alkali metal (with n=1) or an alkaline earth metal (with n=2).

21: A compound of formula (VI) 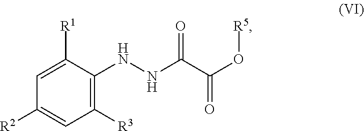
wherein R¹ and R³ are defined according to claim 1,
wherein R¹ and R³ are not simultaneously hydrogen in any compound, R² is halogen-substituted C₁-C₄-alkyl or halogen-substituted C₁-C₄-alkoxy and R⁵ is C₁-C₄-alkyl.

22: A compound of formula (IVa) or (IVb) 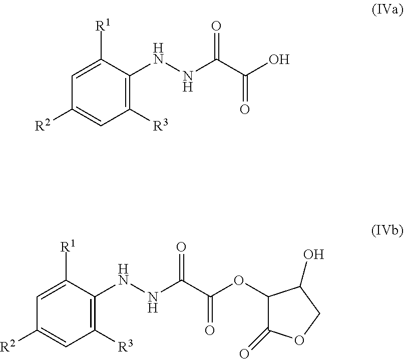
wherein R¹ and R³ are defined according to claim 1,
wherein R¹ and R³ are not simultaneously hydrogen in any compound and R² is halogen-substituted C₁-C₄-alkyl or halogen-substituted C₁-C₄-alkoxy.
